(19) United States
(12) Patent Application Publication
(10) Pub. No.: US 2025/0031688 A1
(43) Pub. Date: Jan. 30, 2025

(54) METHODS AND COMPOSITIONS FOR CRYOPRESERVATION OF IMMUNE CELLS

(71) Applicant: TAKEDA PHARMACEUTICAL COMPANY LIMITED, Osaka (JP)

(72) Inventors: Shuxia ZHOU, Cambridge, MA (US); Yana WANG, Cambridge, MA (US); Lan CAO, Cambridge, MA (US); Dayong GAO, Cambridge, MA (US); Qiong XUE, Cambridge, MA (US); Jiusong SUN, Cambridge, MA (US); Huang ZHU, Cambridge, MA (US)

(21) Appl. No.: 18/361,790

(22) Filed: Jul. 28, 2023

(51) Int. Cl. A01N 1/02; A61K 39/00; C07K 14/54; C07K 14/705; C07K 14/725; C07K 16/28

(52) U.S. Cl. CPC A01N 1/0221; A61K 39/4613; A61K 39/4631; C07K 14/5443; C07K 14/7051; C07K 14/70521; C07K 16/2803; A61K 2239/21; A61K 2239/22; C07K 2317/622

(57) ABSTRACT

The present disclosure provides, among other things, a cryopreservation medium comprising a cryoprotectant, an albumin, a disaccharide and a non-pyrogenic and isotonic crystalloid solution. The disclosure also provides, among other things, a cryopreservation medium for cryopreserving immune cells, the medium comprising: human serum albumin (HSA), sodium chloride, sodium gluconate, sodium acetate trihydrate, potassium chloride, magnesium chloride, dimethyl sulfoxide (DMSO), and a trehalose. The present disclosure also provides, a method of cryopreserving immune cells, transporting and subsequently administering such immune cells to a patient in need thereof.

### INCORPORATION BY REFERENCE OF SEQUENCE LISTING

The present specification makes reference to a Sequence Listing submitted electronically as a ST26 XML file named “MIL-009US2_SL”. The XML file was generated on Jul. 28, 2023 and is 1,048,576 bytes in size. The entire contents of the Sequence Listing are herein incorporated by reference in their entirety.

### BACKGROUND

Freezing of cells has long been used to preserve living cells after the cells have been removed, harvested or separated from a donating organism or from a cell culture. However, the cryopreservation and recovery of living cells remains challenging. This is believed to be, in part, because cells subjected to freezing and thawing conditions are exposed to harsh conditions which in turn result in a generally low survivability and/or viability rate.

Freezing is destructive to most living cells. Generally, as the extracellular medium freezes, cells attempt to maintain osmotic equilibrium across the membrane leading to intracellular water loss, which in turn increases intracellular solute concentration until intracellular freezing occurs. It is believed that both intracellular freezing and solution effects are responsible for cell injuries. Such cell injuries include, for example, damage to cells plasma membrane which results from osmotic dehydration of the cells.

### SUMMARY

The present application is based, at least in part, on the discovery of a cryopreservation media for immune cells, which enables high survivability and/or viability and is particularly useful for cryopreservation of adoptive cell therapies.

In some embodiments, a cryopreservation medium described herein comprises a non-pyrogenic and isotonic crystalloid solution, a disaccharide, a cryoprotectant and an albumin.

In various embodiments, the non-pyrogenic and isotonic crystalloid solution is present at a concentration of 25% v/v to 50% v/v. In some embodiments, the non-pyrogenic and isotonic crystalloid solution is present at a concentration of about 25%, 30%, 35%, 40%, 45%, or 50%.

In some embodiments, the disaccharide is selected from the group consisting of sucrose, lactose, maltose, trehalose, cellobiose, and chitobiose. Accordingly, in some embodiments, the disaccharide is sucrose. In some embodiments, the disaccharide is lactose. In some embodiments, the disaccharide is cellobiose. In some embodiments, the disaccharide is lactose. In some embodiments, the disaccharide is chitobiose. In a particular embodiment, the disaccharide is trehalose.

In some aspects, a cryopreservation medium provided herein comprises sodium chloride, potassium chloride, magnesium chloride hexahydrate, sodium acetate trihydrate, sodium gluconate, adenosine, dextran-40, lactobionic acid, HEPES, sodium hydroxide, L-glutathione, potassium chloride, potassium bicarbonate; potassium phosphate, dextrose, sucrose, mannitol, calcium chloride dihydrate, magnesium chloride, sodium hydroxide, potassium hydroxide, DMSO, human serum albumin and trehalose.

In some embodiments, a cryopreservation medium provided herein comprises about 2.5% v/v human serum albumin (I-ISA). In some embodiments, the cryopreservation medium comprises between about 2.5% v/v and 5.0% v/v human serum albumin (HSA). In some embodiments, the cryopreservation medium comprises between about 2.0% v/v and 3.0% v/v human serum albumin (HSA).

In some embodiments, the cryopreservation medium comprises between about 10 mM-100 mM trehalose. In some embodiments, the cryopreservation medium comprises between about 10 mM-50 mM trehalose. In some embodiments, the cryopreservation medium comprises between about 20 mM-40 mM trehalose. In some embodiments, the cryopreservation medium comprises about 30 mM trehalose.

In some embodiments, a cryopreservation medium is provided comprising: human serum albumin (HSA), sodium chloride, sodium gluconate, sodium acetate trihydrate, potassium chloride, magnesium chloride, dimethyl sulfoxide (DMSO), and a trehalose.

In some embodiments, the ISA is at a concentration of between about 1.25% v/v to 15% v/v, and the trehalose is at a concentration of between about 10 mM-100 mM.

In some embodiments, the HSA is at a concentration of about 10% v/v. In some embodiments, the trehalose is at a concentration of about 30 mM.

In some aspects, a cryopreservation medium suitable for immune cells is provided, the medium comprising: PlasmaLyte-A, human serum albumin (HSA), trehalose and a cryoprotectant.

In some embodiments, the cryoprotectant is DMSO.

In some embodiments, the cryopreservation medium further comprises 0-55 mM HEPES, and 0-6% v/v dextran.

In some embodiments, the cryopreservation medium further comprises a sugar alcohol, dextran, a metabolite, and an anti-oxidant.

In some embodiments, the sugar alcohol is mannitol at a concentration between 0-100 mM. In some embodiments, mannitol is at a concentration of between about 10-80 mM. In some embodiments, mannitol is at a concentration of between about 20-70 mM. In some embodiments, mannitol is at a concentration of between about 30-60 mM.

In some embodiments, the metabolite is adenosine.

In some embodiments, the anti-oxidant is glutathione.

In some embodiments, the medium is suitable for cryopreserving natural killer (NK) cells.

In some embodiments, the NK cells are cord blood derived or induced pluripotent stem cell (iPSC) derived NK cells. Accordingly, in some embodiments, the NK cells are cord blood derived. In some embodiments, the NIK cells are iPSC derived NK cells.

In some embodiments, the NK cells are genetically engineered cord blood NK cells.

In some embodiments, the NK cells are genetically engineered with a chimeric antigen receptor (CAR).

In some embodiments, the CAR binds CD19.

In some embodiments, the genetically engineered cord blood NK cells comprise human cord blood-derived NK cells (CB-NK) transduced with a retroviral vector expressing an iCaspase9, a CD19-CAR and an IL-15. In some embodiments, the genetically engineered cord blood NK cells comprise human cord blood-derived NK cells (CB-NK) transduced with non-viral vector expressing an iCaspase9, a CD19-CAR and an IL-15.

In some embodiments, the genetically engineered cord blood NK cells include a CD19-CAR comprising an anti-CD19 binding domain, a transmembrane domain such as the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154 and an intracellular signaling domain such as an intracellular signaling domain FcR gamma, FcR beta, CD3 gamma, CD3 delta, CD3-zeta, CD3 epsilon, CD5, CD22, CD79a, CD79b, and CD66d. The CD-19 binding domain can be a single chain antibody or single chain antibody fragment, such as an scFv. In one embodiment, the anti-CD19 binding domain comprises a light chain variable region comprising the amino acid sequence set forth in SEQ ID NO: 1 and/or a heavy chain variable region comprising the amino acid sequence set forth in SEQ ID NO: 2. In another embodiment, the CD-19 CAR can include an anti-CD19 binding domain, a CD28 transmembrane domain (an exemplary CD28 transmembrane sequence is shown in SEQ ID NO: 3, a CD3z signaling domain (an exemplary CD3z sequence is shown in SEQ ID NO: 4 and can further include a suicide switch such as iCaspase9 and/or IL-15.

In one embodiment, the genetically engineered cord blood NK cells include a nucleic acid molecule encoding the heavy chain variable region of an anti-CD19 binding domain and/or a nucleic acid molecule encoding the light chain variable region of an anti-CD19 binding domain.

In some embodiments, the genetically engineered cord blood NK cells are present at a concentration of between 6 M/mL to 120 M/mL. In some embodiments, the genetically engineered cord blood NK cells are present at a concentration of between 6 M/mL to 200 M/mL. In some embodiments, the genetically engineered cord blood NK cells are present at a concentration of between 6 M/mL to 25 M/mL. In some embodiments, the genetically engineered cord blood NK cells are present at a concentration of between 6 M/mL to 120 M/mL in a volume of medium ranging from 30-45 mLs. In some embodiments, the genetically engineered cord blood NK cells are present at a concentration of between 6 M/mL to 200 M/mL in a volume of medium ranging from 30-45 mLs. In some embodiments, the genetically engineered cord blood NK cells are present at a concentration of between 6 M/mL to 25 M/mL in a volume of medium ranging from 30-45 mLs.

In some embodiments, CAR-NK cells are formulated in a cryopreserved media provided herein at a concentration ranging from 100 million cells to 900 million cells, present in a volume of medium ranging from 30-45 mLs. In a particular embodiment, CAR-NK cells are present at a concentration of about 200 million cells in a volume of about 36 mLs of media. In another embodiment, CAR-NK cells are present at a concentration of about 800 million cells in a volume of about 36 mLs of media. In some embodiments, cells in 36 mLs of media are contained in a aseptic container (e.g., an AT vial).

In some aspects, a method of cryopreserving immune cells is provided, the method comprising: (a) contacting immune cells with a cryopreservation medium comprising a cryoprotectant, an albumin, a disaccharide, and a non-pyrogenic and isotonic crystalloid solution; (b) cooling the cells at a controlled rate to a temperature of −80° C.; and (c) storing the cells in liquid nitrogen vapor phase, thereby cryopreserving the immune cells.

In some embodiments, the immune cells are NK cells or T cells.

In some embodiments, the NK cells are freshly isolated or from a cell line.

In some embodiments, wherein the NK cells are derived from cord-blood, peripheral blood, T cells, iPS cells. Accordingly, in some embodiments, the NK cells are derived from cord-blood. In some embodiments, the NIK cells are derived from peripheral blood. In some embodiments, the NK cells are derived from T cells. In some embodiments, the NK cells are derived from iPS cells.

In a particular embodiment, NK cells comprise human cord blood-derived NK cells (CB-NK) transduced with a retroviral vector expressing an iCaspase9, a CD19-CAR and an IL-15. In a particular embodiment, NK cells comprise human cord blood-derived NK cells (CB-NK) transduced with a non-retroviral vector expressing an iCaspase9, a CD19-CAR and an IL-15.

In some embodiments, the method further comprises thawing the cryopreserved immune cells (e.g., NK cells). In a particular embodiment, the cryopreserved immune cells are genetically engineered cord blood NK cells comprising a CD19-CAR comprising a light chain variable region comprising the amino acid sequence set forth in SEQ ID NO: 1 and/or a heavy chain variable region comprising the amino acid sequence set forth in SEQ ID NO: 2. In another embodiment, the CD-19 CAR can include an anti-CD19 binding domain, a CD28 transmembrane domain (an exemplary CD28 transmembrane sequence is shown in SEQ ID NO: 3, a CD3z signaling domain (an exemplary CD3z sequence is shown in SEQ ID NO: 4 and can further include a suicide switch such as iCaspase9 and/or IL-15. In one embodiment, the genetically engineered cord blood NK cells include a nucleic acid molecule encoding the heavy chain variable region of an anti-CD19 binding domain set forth in SEQ ID NO: 2 and/or a nucleic acid molecule encoding the light chain variable region of an anti-CD19 binding domain set forth in SEQ ID NO: 1.

In some embodiments, the once-cryopreserved and thawed NK cells maintain cellular activity and function comparable or similar to freshly isolated NK cells that have not been frozen.

In some embodiments, the thawed NK cells are suitable for therapeutic use. In a particular embodiment, the NK cells suitable for therapeutic use are CAR-NK cells (e.g., cord blood cells transduced with a retroviral vector to express an iCaspase9, a CD19-CAR and an IL-15. In a particular embodiment, the NK cells suitable for therapeutic use are CAR-NK cells (e.g., cord blood cells transduced with a non-viral vector to express an iCaspase9, a CD19-CAR and an IL-15). In some embodiments, thawing the cryopreserved immune cells (e.g., NK cells) comprises: (a) heating a water bath to a temperature ranging from 37° C. and 70° C.; (b) transferring a container comprising cryopreserved immune cells (e.g., NK cells) to the pre-heated water bath; and (b) agitating the container at a speed of between about 100 and about 250 RPM for a suitable period of time, thereby thawing the immune cells (e.g., NK cells). In some embodiments, the temperature is between about 55° C. and 65° C. In some embodiments, the agitating speed is between about 100 and 125 RPM. In some embodiments, the thawing media is agitated at the recited RPM.

In some embodiments, thawing the cryopreserved immune cells (e.g., NK cells) comprises: (a) heating a dry heating device to a temperature ranging from 37° C. and 90° C.; (b) transferring a container comprising cryopreserved immune cells (e.g., NK cells) to the dry heating device; and (b) agitating the container at a speed of between about 100 and about 250 RPM for a suitable period of time, thereby thawing the immune cells (e.g., NK cells). In some embodiments, the temperature is between about 55° C. and 65° C. In some embodiments, the temperature is between about 65° C. and 90° C. In some embodiments, the agitating is between about 100 and 125 RPM. In some embodiments, the thawing media is agitated at the recited RPM.

In some aspects, a cell therapy product (e.g., a CAR-NK cell therapy product) is provided for administration to a subject in need thereof comprising: (a) a population of engineered NK cells comprising cord blood NK cells transduced with a retroviral vector expressing anti-CD19 chimeric antigen receptor (CAR), IL-15, and iCaspase9; and (b) a cryopreservation medium as described herein. In some embodiments, a cell therapy product suitable for administration to a subject in need thereof comprises a population of CAR-NK cells expressing anti-CD19 chimeric antigen receptor (CAR), IL-15, and iCaspase9 formulated in a cryopreservation medium comprising a cryoprotectant, a disaccharide, an albumin and a non-pyrogenic and isotonic crystalloid solution, wherein the population of cells comprises 200 million CAR-NK cells to 800 CAR-NK cells. In a particular embodiment, a cell therapy product suitable for administration to a subject in need thereof comprises 200-800 million CAR-NK cells expressing anti-CD19 chimeric antigen receptor (CAR), IL-15, and iCaspase9 formulated in a cryopreservation medium comprising PlasmaLyte-A, trehalose, CS10 and HSA. In some aspects, a population of CD19 chimeric antigen receptor (CAR) NK cells in a cryopreservation medium is provided as described herein.

In some embodiments, the CD19-CAR NK cells comprise IL-15.

In some embodiments, the CD19-CAR NK cells comprise iCaspase9.

In some aspects, a cell therapy product is provided comprising a population of CAR-NK cell comprising cord blood NK cells genetically modified to express a CD-19 CAR, an iCaspase and an IL-15 formulated in a cryopreservation medium comprising PLASMA-LYTE A, trehalose, CS10 and HSA.

In some embodiments, the concentration of cells in the product is between about 6 million cells/mL and 200 million cells/mL. In some embodiments, the concentration of cells in the product is between about 6 million cells/mL and 120 million cells/mL.

In some embodiments, the total viable cells post thawing is between about 200 million to about 800 million cells.

Various aspects of the invention are described in detail in the following sections. The use of sections is not meant to limit the invention. Each section can apply to any aspect of the invention. In this application, the use of “or” means “and/or” unless stated otherwise. As used herein, the singular forms “a”, “an”, and “the” include both singular and plural referents unless the context clearly dictates otherwise.

### BRIEF DESCRIPTION OF THE DRAWINGS

FIG. 1 is a table that includes description of cryopreservation media and in vivo experimental summary for chimeric antigen receptor (CAR) NK cryopreservation formulation screening study (i.e., “CAR-NK”) cells.

FIG. 2A is a graph that shows the survival of animals following administration of tumor cells and NK cells that formulated in exemplary cryopreservation media described herein. FIG. 2B shows a series of bioluminescent imaging (BLI) pictures that shows NK cell ability to control tumor following administration into mouse models. FIG. 2C summarizes the median survival of animals that were administered NK cells formulated in exemplary cryopreservation media and the statistical analysis.

FIG. 3A depicts exemplary cryoformulations for a 2^{nd} in vivo cryopreservation media study. FIG. 3B is a graph that shows the survival curve and. FIG. 3C shows a graph that demonstrates tumor control by NK cells as represented by the total radiance flux. FIG. 3D summarizes the median survival of animals that were administered NK cells and the statistical analysis. FIG. 3E is a series of BLI pictures that shows tumor control by NK cells following administration into mouse models.

FIG. 4A-4D depict a series of graphs that show in vivo efficacy of NK cells that were previously preserved or formulated in a composition comprising 40% PLASMA-LYTE A+50% CS10+10% HSA+30 mM trehalose. FIG. 4A and FIG. 4B represent CARNK cells from 2 different donors, while FIG. 4C and FIG. 4D represent CAR NK cells from the same donor but formulated into 2 different containers. The data from these graphs show that the once-cryopreserved NK cells from all 3 donors showed in vivo efficacy while 2 out of 3 donors have in vivo efficacy comparable to that observed with fresh cells. FIGS. 4E and 4F is a series of BLI pictures from mice that had either received once-frozen NK cells in a composition comprising 40% PLASMA-LYTE A+50% CS10+10% HSA+30 mM trehalose, or freshly harvested cells. The data from these BLI studies confirms the in vivo efficacy of cryopreserved NK cells using composition comprising 40% PLASMA-LYTE A+50% CS10+10% HSA+30 mM trehalose. FIG. 4G, FIG. 4H and FIG. 4I is a series of graphs that show tumor control by NK cells as represented by the total radiance flux.

FIGS. 5A and 5D depicts a series of graphs that show comparable in vitro function (i.e., % killing) of CAR-NK cells using cryopreservation media described herein. FIG. 5B and FIG. 5E are the table summarized tested conditions and cytotoxicity results. FIG. 5C, and FIG. 5F are a series of tables that show comparable phenotype of CAR-NK cells using cryopreservation media described herein.

FIG. 6 depicts a series of graphs that show comparable in vitro parameters of NK cells from 4 different donors that were prepared at a concentration of 10 million/mL in 2 mL cryovials and stored in a composition comprising 40% PLASMA-LYTE A+50% CS10+10% HSA+30 mM trehalose relative to fresh cells.

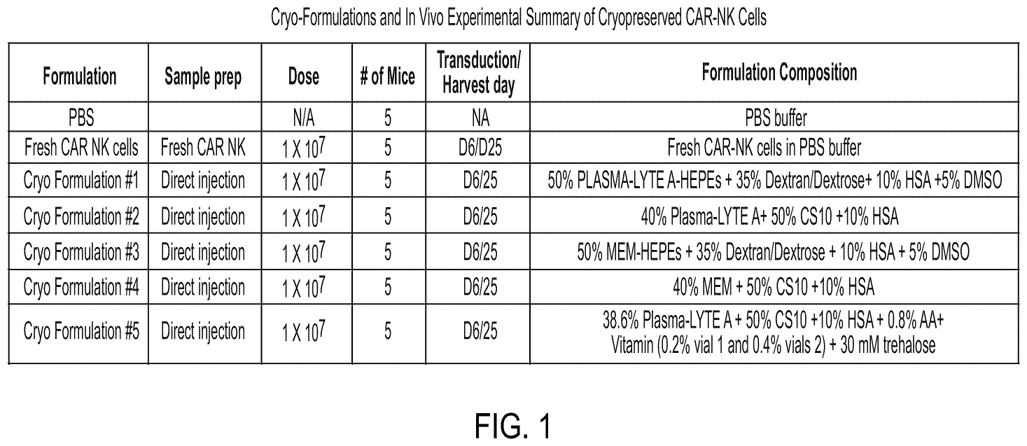

### DEFINITIONS

Administering: As used herein, the terms “administering,” or “introducing” are used interchangeably in the context of delivering a once-frozen cell of interest or a population of such cells to a patient in need thereof. Various methods are known in the art for administering cells to patients, including for example administering the cells to a patient in need thereof by intravenous or surgical methods.

Adoptive Cell Therapy: As used herein interchangeably, the terms “adoptive cell therapy” or “adoptive cell transfer” or “cell therapy” or “ACT” refer to the transfer of cells, for example, a population of genetically modified cells, into a patient in need thereof. The cells can be derived and propagated from the patient in need thereof (i.e., autologous cells) or could have been obtained from a non-patient donor (i.e., allogeneic cells). In some embodiments, the cell is an immune cell, such as a lymphocyte. Various cell types can be used for ACT including but not limited to, natural killer (NK) cells, T cells, CD8+ cells, CD4+ cells, delta-gamma T-cells, regulatory T-cells, induced pluripotent stem cells (iPSCs), iPSC derived T cells, iPSC derived NK cells, hematopoietic stem cells (HSCs), mesenchymal stem cells (MSCs) and peripheral blood mononuclear cells. Furthermore, the cells that have been cryopreserved using the compositions and/or methods described herein may either be genetically modified prior to being cryopreserved or be genetically modified after they have been cryopreserved and thawed. The cells for adoptive cell therapy cryopreserved using compositions and methods described herein retain high viability and suitability for ACT applications. For example, in some embodiments, the cells that have been cryopreserved using methods and compositions described herein are genetically modified to introduce a chimeric antigen receptor (CAR) after they are thawed. Alternatively, in some embodiments, a cell that has already been previously genetically modified (e.g., a CAR-T or CAR-NK cell) can be cryopreserved using the cryopreservation medium and/or methods described herein and retain high survivability and suitability for ACT applications.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a stated value of interest as well as value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Allogeneic: As used herein, allogeneic refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically

Autologous: As used herein, the term “autologous” means from the same individual. For example, “autologous” in relation to donor and recipient means that the donor subject is the recipient subject.

Chimeric Antigen Receptor (CAR): As used herein, the term “chimeric antigen receptor” or “CAR” engineered receptors which can confer an antigen specificity onto cells (for example immune cells such as NK cells, iPSC derived NK cells (iNK cells), T cells such as naive T cells, central memory T cells, effector memory T cells, gamma delta T cells, T regulatory cells or combinations thereof). CARs are also known as artificial T-cell receptors, chimeric T-cell receptors or chimeric immunoreceptors. In various embodiments, a CAR described herein may include one or more of an antigen-specific targeting domain, an extracellular domain, a transmembrane domain, optionally one or more co-stimulatory domains, and an intracellular signaling domain.

Cells: As used herein, the term “cells” refers to any cells that can be subjected to cryopreservation. In some embodiments, the cells are a stem cell or progenitor cell. In certain embodiments, the cells are somatic cells, e.g., adult stem cell, progenitor cell, or differentiated cell. In some embodiments, the cells are hematopoietic cell, e.g., a hematopoietic stem or progenitor cell. In some embodiments, the cells include B-cells, T cells, monocytes or progenitor cells. In some embodiments, the cells are NK cells, and in particular CAR-NK cells.

Controlled Cooling or Cooling at a Controlled Rate: The terms “controlled cooling” or “cooling at a controlled rate” and similar terms as used herein, is a process which applies an external cooling regime that results in a decrease in temperature of a biological sample cooled at a rate between for example, 0.1° C./minute and 50° C./minute. In some embodiments, the controlled cooling can be achieved using a commercially available freezer such as a controlled rate freezer. Various examples of controlled rate freezers and include, for example, but not limited to, CryoMed™ Model 5474, CryoMed™ Model 5478, Strex CytoSensei SB02-0920, Custom BioGenic Systems Model 2101.

Cryopreservation: As used herein, the term “cryopreservation” generally refers to a freezing a biological material (e.g., a population of cells) to low enough temperatures, such that chemical processes, which might otherwise damage the material are halted thereby preserving the material. Cryopreserved cells maintain viability for an extended period of time in the frozen state, such as for 1, 5, 10 or more years in the cryopreserved state. The cryopreserved cells, once thawed, are able to propagate both for in vitro and in vivo applications.

Cryoprotectant: As used herein, the term “cryoprotectant” means a substance used to protect biological tissue from freezing damage. Exemplary cryoprotectants include, for example, dimethyl sulfoxide (DMSO), glycerol, ethylene glycol and propanediol.

Crystalloid: As used herein, the term “crystalloid” means a composition comprising mineral salts in an aqueous solution with or without other water-soluble molecules, such as buffers.

Ex vivo: As used herein, the term “ex vivo” means a process in which cells are removed from a living organism and are propagated outside the organism (e.g., in a test tube, in a culture bag, in a bioreactor).

Fresh cell or Rescued Fresh Cell: As used herein, the terms “fresh,” “fresh cell,” or “rescued fresh cell” refers to mammalian cells that have never been frozen and/or once frozen but subsequently restimulated, cultured in culture medium and then harvested as fresh cells.

Functional equivalent or derivative: As used herein, the term “functional equivalent” or “functional derivative” denotes, in the context of a functional derivative of an an amino acid sequence or any other molecule (e.g., a media formulation component) that retains an activity (either function or structural) that is substantially similar to that of the original molecule or sequence. A functional derivative or equivalent may be a natural derivative or is prepared synthetically. Exemplary derivatives include those having chemico-physical properties which are similar to that of the original molecule or sequence. Desirable similar chemico-physical properties include, similarities in charge, bulkiness, hydrophobicity, hydrophilicity, and the like.

Isotonic: As used herein, the term “isotonic” means having an osmotic pressure that is equal to or approximately the same as the osmotic pressure of a physiological fluid.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Latent Heat or Latent Heat of Fusion: The terms “Latent Heat of Fusion” and “latent heat” as used in its broadest sense is indicative of any substance or phenomenon wherein, as heat is applied to the substance at a substantially uniform rate in the process of fusing the same, a point is reached where the temperature of the substance temporarily ceases to rise while heat is being absorbed for the modification of the molecular structure and internal energy of the substance. During freezing, release of the latent heat during phase change from liquid to solid state increases the temperature of the surroundings, leading to cessation of vitrification. In some embodiments, the latent heat of fusion can cause melting of ice. In some embodiments, the melting of ice causes the concentration of the sugars, salts, and cryoprotectant (e.g. DMSO or glycerol) to increase, and, consequently, also causes the osmotic pressure of the unfrozen fraction, to increase rapidly. In some embodiments, the increase in the osmotic strength causes an efflux of water from cells. In some embodiments, as cooling continues, these processes continue until the viscosity of the unfrozen fraction becomes too high for any further crystallization.

Primary Cell: The term, “primary cell,” refers to cells that are directly isolated from a subject and which are subsequently propagated.

Polypeptide: The term, “polypeptide,” as used herein refers a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Storage Temperature: The term “storage temperature”, as used herein, refers to the temperature at which the cells are stored. In some embodiments, the cells are stored in liquid nitrogen vapor phase. In some embodiments, the cells are stored at a temperature below −60° C. In another embodiment, the cells are stored at a temperature ranging from −60° C. to −140° C. In another embodiment, the cells are stored at a temperature ranging from −60° C. to −196° C. In some embodiments, the cells are stored at or below a temperature of −140° C. In some embodiments, the cells are stored at temperature below −196° C.

Shipping Temperature: The term “shipping temperature” as used herein refers to the temperature at which the cells are shipped or transported, e.g., from a first location where the cells may be manufactured and/or cryopreserved to a second location where the cells may be thawed and subsequently administered to a subject in need thereof. In some embodiments, the cells are shipped in liquid nitrogen vapor phase. In some embodiments, the cells are shipped at a temperature of −140° C. or below −140° C. In some embodiments, the cells are stored and/or shipped at a temperature of −140° C. or below −140° C.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of the disease, disorder, and/or condition.

Sugar or Saccharide: The terms “sugar” and “saccharide” herein have been used interchangeably, and generally refer to oligosaccharides such as monosaccharides, disaccharides, trisaccharides or polysaccharides, and the like. In some embodiments, the saccharide is one or more of glucose, xylose, arabinose, fructose, galactose, mannose, mannitol, sorbitol, xylitol, myoinositol, trehalose, sucrose, lactose, maltose, cellobiose, lactitol, maltitol, methyl cellulose, carboxymethyl cellulose, dextran, glycogen, amylose, amylopectin, inulin, sodium alginate, ethyl cellulose, hydroxyethyl cellulose, raffinose, stachyose, xanthan gum, glucosamine, and galactosamine. In some embodiments, saccharide is a disaccharide. In some embodiments, the disaccharide is sucrose, lactose, maltose, trehalose, cellobiose, or chitobiose. In some other embodiments, the disaccharide is trehalose. In some embodiments, one or more sugars includes trehalose, sucrose, mannitol, and/or dextran.

Sugar Alcohol: The term “sugar alcohol” as used herein refers to a hydrocarbon having between about 4 and about 8 carbon atoms and a hydroxyl group. In some embodiments, the sugar alcohol includes mannitol, sorbitol, inositol, galactitol, dulcitol, xylitol, or arabitol.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose. In some embodiments, a therapeutically effective amount of an adoptive cell therapy, as used herein, is a dosage of cells (e.g., a population of genetically modified immune cells such as CAR-T or CAR-NK) in a certain formulation (e.g., a cryopreservation media described herein) administered to a subject in need thereof (e.g., a patient suffering from a B-cell malignancy). For example, in some embodiments, a therapeutically effective amount comprises CAR-NK cells at a concentration of between 6 M/mL to 120 M/mL in a volume between 10 mL and 45 mL. In some embodiments, a therapeutically effective amount comprises CAR-NK cells at a concentration of between 5M/mL to 25M/mL in a volume between 10 mL and 45 mL. In some embodiments, a therapeutically effective amount comprises CAR-NK cells at an amount of about 200 million cells to about 800 million cells. In a particular embodiment, the CAR-NK cells have been genetically modified to express an iCaspase, an IL-15 and a CD-19 chimeric antigen receptor.

In a particular embodiment, the CAR-NK cells have been genetically modified to express a CD-19 CAR comprising a heavy chain variable region comprising the amino acid sequence set forth in SEQ ID NO:2 or a sequence having at least 95% identity to the sequence set forth in SEQ ID NO: 2 and/or a light chain variable region comprising the amino acid sequence set forth in SEQ ID NO: 1 or a sequence having at least 95% identity to the amino acid sequence set forth in SEQ ID NO: 1.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

The recitation of numerical ranges by endpoints herein includes all numbers and fractions subsumed within that range (e.g. 1 to 5 includes 1, 1.5, 2, 2.75, 3, 3.9, 4 and 5). It is also to be understood that all numbers and fractions thereof are presumed to be modified by the term “about.”

Various aspects of the invention are described in detail in the following sections. The use of sections is not meant to limit the invention. Each section can apply to any aspect of the invention. In this application, the use of “or” means “and/or” unless stated otherwise. As used herein, the singular forms “a”, “an”, and “the” include both singular and plural referents unless the context clearly dictates otherwise.

### DETAILED DESCRIPTION

### Cryopreservation Media

Provided herein are various media suitable for freezing or cryopreserving cells and in particular, immune cells. Various immune include but are not limited to, for example, NK cells, T-cells (e.g., alpha-beta T cells, gamma-delta T cells, regulatory T cells), B cells, MSCs, HSCs, iPSC derived NK and iPSC derived T cells. In one embodiment, the cells are NK cells, in particular, allogeneic NK cells expressing a CAR (i.e., CAR-NK cells).

In some aspects, a suitable medium for cryopreserving (i.e., a cryopreservation medium) cells comprises: a cryoprotectant, an albumin, a disaccharide and a non-pyrogenic and isotonic crystalloid solution. In various embodiments, cells once cryopreserved in a media described, can be thawed and subsequently administered to a patient, without the need to reformulate or resuspend the cells in another media or solution.

Various cryoprotectants are known in the art and include, for example, dimethyl sulfoxide (DMSO), glycerol, and propanediol among others. In some embodiments, the cryopreservation medium comprises DMSO as a cryoprotectant.

In some embodiments, human serum albumin (HSA) is the albumin in the cryopreservation medium.

In some embodiments, the suitable cryopreservation medium also comprises a saccharide or a sugar. In another aspects, a suitable cryopreservation medium described herein comprises: HSA, sodium, sodium chloride, sodium gluconate, sodium acetate trihydrate, potassium chloride, magnesium chloride, dimethyl sulfoxide (DMSO), and a disaccharide.

In some embodiments, a saccharide includes a monosaccharide, a disaccharide, a trisaccharide or a polysaccharide. In some embodiments, the saccharide is a disaccharide. In some embodiments, the disaccharide is sucrose, lactose, maltose, trehalose, cellobiose, or chitobiose. Accordingly, in some embodiments, the disaccharide is sucrose. In some embodiments, the disaccharide is lactose. In some embodiments, the disaccharide is maltose. In some embodiments, the disaccharide is trehalose. In some embodiments, the disaccharide is cellobiose. In some embodiments, the disaccharide is chitobiose.

In some embodiments, a suitable cryopreservation medium incudes one or more of glucose, xylose, arabinose, fructose, galactose, mannose, mannitol, sorbitol, xylitol, myoinositol, trehalose, sucrose, lactose, maltose, cellobiose, lactitol, maltitol, methyl cellulose, carboxymethyl cellulose, dextran, glycogen, amylose, amylopectin, inulin, sodium alginate, ethyl cellulose, hydroxyethyl cellulose, raffinose, stachyose, xanthan gum, glucosamine, and galactosamine. In some embodiments, a suitable cryopreservation medium includes trehalose, sucrose, mannitol, and/or dextran. In some embodiments, a suitable cryopreservation medium incudes one or more sugars selected from trehalose, sucrose and/or mannitol.

Various concentration of saccharides or sugar can be used in a cryopreservation medium. In some embodiments, the cryopreservation medium includes trehalose, sucrose, or mannitol between about 0 mM-500 mM. In some embodiments, the cryopreservation medium includes trehalose, sucrose, or mannitol between about 0 mM-200 mM. In some embodiments, the cryopreservation medium includes trehalose, sucrose, or mannitol between about 0 mM-100 mM.

In some embodiments, the cryopreservation media includes one or more sugars selected from trehalose, sucrose and/or mannitol at a concentration of between about 0-100 mM. In some embodiments, the cryopreservation medium includes mannitol between about 0-100 mM.

In some embodiments, the cryopreservation medium includes trehalose between about 10 mM-100 mM. In some embodiments, the cryopreservation medium includes 30 mM trehalose.

Accordingly, in some embodiments, trehalose, sucrose, or mannitol is present in the cryopreservation medium at a final concentration of about 1 mM, 5 mM, 10 mM, 15 mM, 20 mM, 25 mM, 30 mM, 35 mM, 40 mM, 45 mM, 50 mM, 55 mM, 60 mM, 65 mM, 70 mM, 75 mM, 80 mM, 85 mM, 90 mM, 95 mM, 100 mM, 105 mM, 110 mM, 115 mM, 120 mM, 125 mM, 130 mM, 135 mM, 140 mM, 145 mM, 150 mM, 155 mM, 160 mM, 165 mM, 170 mM, 175 mM, 180 mM, 185 mM, 190 mM, 195 mM, or 200 mM.

Accordingly, in some embodiments, trehalose, sucrose, or mannitol is present in the cryopreservation medium at a final concentration of less than 1 mM, less than 10 mM, less than 20 mM, less than 30 mM, less than 40 mM, less than 50 mM, less than 60 mM, less than 70 mM, less than 80 mM, less than 90 mM, less than 100 mM, less than 110 mM, less than 120 mM, less than 130 mM, less than 140 mM, less than 150 mM, less than 160 mM, less than 170 mM, less than 180 mM, less than 190 mM, or less than 200 mM.

In some embodiments, the cryopreservation medium includes a dextran. In some embodiments, the cryopreservation medium includes a dextran between about 0-20 w/v %. In some embodiments, the cryopreservation medium includes a dextran between about 0-6 w/v %. Accordingly, in some embodiments, dextran is present in the cryopreservation medium at a final concentration of about 0.2 w/v %, 0.4 w/v %, 0.6 w/v %, 0.8 w/v %, 1.0 w/v %, 1.5 w/v %, 2.0 w/v %, 2.5 w/v %, 3.0 w/v %, 3.5 w/v %, 4.0 w/v %, 4.5 w/v %, 5.0 w/v %, 5.5 w/v %, 6.0 w/v %, 6.5 w/v %, 7.0 w/v %, 7.5 w/v %, 8.0 w/v %, 8.5 w/v %, 9.0 w/v %, 9.5 w/v %, 10.0 w/v %, 10.5 w/v %, 11.0 w/v %, 11.5 w/v %, 12.0 w/v %, 12.5 w/v %, 13.0 w/v %, 13.5 w/v %, 14.0 w/v %, 14.5 w/v %, 15.0 w/v %, 15.5 w/v %, 16.0 w/v %, 16.5 w/v %, 17.0 w/v %, 17.5 w/v %, 18.0 w/v %, 18.5 w/v %, 19.0 w/v %, 19.5 w/v %, or 20.0 w/v %.

In some embodiments, the cryopreservation medium comprises a non-pyrogenic and isotonic crystalloid solution. Various non-pyrogenic and isotonic crystalloid solutions can be used in the cryopreservation medium described herein. For example, exemplary isotonic crystalloid solutions that may be used in the cryopreservation media described herein include PLASMA-LYTE A, normal saline, a lactate buffered solution, an acetate buffered solution, an acetate and lactate buffered solution, and a dextrose in water solution. Generally, non-pyrogenic and isotonic crystalloid solutions comprise one or more of the following sodium chloride, sodium gluconate, sodium acetate trihydrate, potassium chloride, and magnesium chloride. Accordingly, in some embodiments, the cryopreservation medium comprises one or more of the following sodium chloride, sodium gluconate, sodium acetate tryhydrate, potassium chloride, and magnesium chloride. In some embodiments, the cryopreservation medium comprises one or more of sodium chloride, sodium gluconate, sodium acetate tryhydrate, potassium chloride, and magnesium chloride. An example of a commercially available non-pyrogenic and isotonic crystalloid solutions is PLASMA-LYTE A (Baxter International Inc.). In some embodiments, the non-pyrogenic and isotonic crystalloid solution is PLASMA-LYTE A. In some embodiments, the non-pyrogenic and isotonic crystalloid solution is a 0.9% normal saline solution. In some embodiments, the non-pyrogenic and isotonic crystalloid solution is a lactate buffered solution. In some embodiments, the non-pyrogenic and isotonic crystalloid solution is a dextrose in water solution.

PLASMA-LYTE A includes sodium chloride, sodium gluconate, sodium acetate trihydrate, potassium chloride, and magnesium chloride. In some embodiments, the cryopreservation medium includes between about 10% v/v-75% v/v PLASMA-LYTE A. In some embodiments, the cryopreservation medium includes between about 25% v/v-50% v/v PLASMA-LYTE A. In some embodiments, the cryopreservation medium includes between about 40% v/v PLASMA-LYTE A. Accordingly, in some embodiments, the cryopreservation medium includes about 10% v/v, 15% v/v, 20% v/v, 25% v/v, 30% v/v, 35% v/v, 40% v/v, 45% v/v, 50% v/v, 55% v/v, 60% v/v, 65% v/v, 70% v/v, or 75% v/v PLASMA-LYTE A.

In some embodiments, the cryopreservation medium includes sodium chloride between about 0.1 mg/mL to about 1 mg/mL. In some embodiments, the cryopreservation medium includes sodium chloride between about 0.4 mg/mL to about 0.6 mg/mL. Accordingly, in some embodiments, the cryopreservation medium includes about 0.1 mg/nL, 0.15 mg/nL, 0.2 mg/mL, 0.25 mg/mL, 0.3 mg/mL, 0.35 mg/mL, 0.4 mg/mL, 0.45 mg/mL, 0.5 mg/mL, 0.55 mg/mL, 0.6 mg/mL, 0.65 mg/mL, 0.7 mg/mL, 0.75 mg/mL, 0.8 mg/mL, 0.85 mg/mL, 0.9 mg/mL, 0.95 mg/mL, 1 mg/mL sodium chloride.

In some embodiments, the cryopreservation medium includes sodium gluconate between about 0.1 mg/mL to about 1 mg/mL. In some embodiments, the cryopreservation medium includes sodium gluconate between about 0.3 mg/mL to about 0.6 mg/mL. Accordingly, in some embodiments, the cryopreservation medium includes about 0.1 mg/mL, 0.15 mg/mL, 0.2 mg/mL, 0.25 mg/mL, 0.3 mg/mL, 0.35 mg/mL, 0.4 mg/mL, 0.45 mg/mL, 0.5 mg/mL, 0.55 mg/mL, 0.6 mg/mL, 0.65 mg/mL, 0.7 mg/mL, 0.75 mg/mL, 0.8 mg/mL, 0.85 mg/mL, 0.9 mg/mL, 0.95 mg/mL, 1 mg/mL sodium gluconate.

In some embodiments, the cryopreservation medium includes between about 25% v/v-75% v/v CS10. In some embodiments, the cryopreservation medium includes between about 40% v/v-60% v/v CS10. In some embodiments, the cryopreservation medium includes about 50% v/v CS10. Accordingly, in some embodiments, the CS10 is present in the cryopreservation medium at about 25% v/v, 30% v/v, 35% v/v, 40% v/v, 45% v/v, 50% v/v, 55% v/v, 60% v/v, 65% v/v, 70% v/v, or 75% v/v. CS10 is available commercially and includes, among other things, dimethyl sulfoxide (DMSO).

In some embodiment, the cryopreservation medium comprises human serum albumin (HSA). In some embodiments, the cryopreservation medium includes between about 0.5 v/v %-25 v/v % HSA. In some embodiments, the cryopreservation medium includes between about 5 v/v %-20 v/v % HSA. In some embodiments, the cryopreservation medium includes about 10 v/v % HSA. In some embodiments, the cryopreservation medium includes about 1.25% v/v to 5% v/v HSA. In some embodiments, the cryopreservation medium includes about 2.5% v/v HSA.

Accordingly, in some embodiments, the cryopreservation medium includes about 0.5 v/v %, 1.0 v/v %, 1.5 v/v %, 2.0 v/v %, 2.5 v/v %, 3.0 v/v %, 3.5 v/v %, 4.0 v/v %, 4.5 v/v %, 5.0 v/v %, 6.0 v/v %, 6.5 v/v %, 7.0 v/v %, 7.5 v/v %, 8.0 v/v %, 8.5 v/v %, 9.0 v/v %, 10.0 v/v %, 10.5 v/v %, 11.0 v/v %, 11.5 v/v %, 12.0 v/v %, 12.5 v/v %, 13.0 v/v %, 13.5 v/v %, 14.0 v/v %, 14.5 v/v %, 15.0 v/v %, 15.5 v/v %, 16.0 v/v %, 16.5 v/v %, 17.0 v/v %, 17.5 v/v %, 18.0 v/v %, 18.5 v/v %, 19.0 v/v %, 19.5 v/v %, 20.0 v/v %, 20.5 v/v %, 21.0 v/v %, 21.5 v/v %, 22.0 v/v %, 22.5 v/v %, 23.0 v/v %, 23.5 v/v %, 24.0 v/v %, 24.5 v/v %, or 25.0 v/v % HSA.

In some embodiments, the cryopreservation medium comprises sodium chloride, potassium chloride, magnesium chloride hexahydrate, sodium acetate trihydrate, sodium gluconate, adenosine, dextran-40, lactobionic acid, HEPES, sodium hydroxide, L-glutathione, potassium chloride, potassium bicarbonate; potassium phosphate, dextrose, sucrose, mannitol, calcium chloride dihydrate, magnesium chloride, sodium hydroxide, potassium hydroxide, DMSO, human serum albumin and trehalose.

In some embodiments, the cryopreservation medium comprises human serum albumin (HSA), sodium chloride, sodium gluconate, sodium acetate trihydrate, potassium chloride, magnesium chloride, dimethyl sulfoxide (DMSO), and a trehalose

In some aspects, the cryopreservation medium comprises one or more of HSA, Ca²⁺, Na⁺, K⁺, Mg²⁺, HEPES, one or more disaccharides, a sugar alcohol, dextran, a metabolite, and an anti-oxidant. In another aspects, the cryopreservation medium comprises: HSA, Na⁺, K⁺, Mg²⁺, HEPES (4-(2-hydroxyethyl)-1-piperazineethanesulfonic acid) at a concentration of about 0-55 mM, one or more sugars selected from trehalose, sucrose and/or mannitol at a concentration of between about 0-100 mM, dextran between about 0-6%, adenosine, and glutathione. In some embodiments, the metabolite is adenosine. In some embodiments, the anti-oxidant is glutathione.

Accordingly, in some embodiments, the HEPES is present in the cryopreservation medium at a concentration of about 0 mM, 0.5 mM, 1 mM, 5 mM, 10 mM, 15 mM, 20 mM, 25 mM, 30 mM, 35 mM, 40 mM, 45 mM, 50 mM, or 55 mM.

In some embodiments, the cryopreservation medium comprises PLASMA-LYTE A, human serum albumin (HSA), trehalose and a cryoprotectant.

In some embodiments, the cryopreservation medium comprises human serum albumin (HSA), PLASMA-LYTE A, a disaccharide and CS10.

In some embodiments, the cryopreservation medium comprises 35-39% PLASMA-LYTE A, 40-50% CS10, 10-20% HSA and 1-5% trehalose.

In some embodiments, the cryopreservation medium comprises 38.6% PLASMA-LYTE A, 50% CS10, 10% HSA and 30 mM trehalose. The cryopreservation medium may further comprises amino acid and/or vitamins.

The cryopreservation media described herein can be formulated in a number of ways.

One embodiment of formulating a cryopreservation medium described herein comprises employing a disaccharide dissolved in PLASMA-LYTE A containing 20% HSA solution. In one embodiment, the formulating is done by mixing the following ratio of components: 1(base media):2 (CS10):1 disaccharide in base media. In some embodiments, the formulation is performed by mixing the following ratio of components: 1(base media):2 (CS10):1 trehalose in base media. In some embodiments, the formulation is performed by mixing the following ratio of components: 1(PLASMA-LYTE A):2 (CS10):1 trehalose in base media.

First, the cells are first suspended in 1 volume of pre-conditioned cold base media (PLASMA-LYTE A containing 20% HSA) to achieve 4× target final cell concentration and then 2 volumes of pre-conditioned cold CS10 solution is added slowly while mixing and keeping the cell suspension cold. In some embodiments, the trehalose solution dissolved in base media is added last to achieve the target cell concentration. In some embodiments, the cells as described herein are added into vials using aseptic technique and then the vials are frozen using the described freezing method.

Another embodiment of formulating a cryopreservation medium described herein comprises: trehalose stock solution in water for injection (WFI). In some embodiments, the formulating is done by mixing the following ratio of components: 1 volume of base media and a disaccharide: 1 volume of CS10. In some embodiments, the formulating is done by mixing the following ratio of components: 1 volume of base media and a trehalose: 1 volume of CS10. In some embodiments, the formulating is done by mixing the following ratio of components: 1 volume of base media containing about 20% HSA and a trehalose: 1 volume of CS10. In some embodiments, the formulating is done by mixing the following ratio of components: 1 volume of PLASMA-LYTE A containing about 20% HSA and a trehalose: 1 volume of CS10

First the cells are suspended in one volume of base media and trehalose to achieve 2× target final cell concentration and then equal volume of CS10 is added while mixing the cells and keeping the cells cold. In some embodiments, the cells as described herein are added into vials using aseptic technique and then the vials are frozen using the described freezing method.

In some aspects, the cryopreservation medium is suitable for cryopreserving natural killer (NK) cells. In some embodiments, the NK cells are from primary cell isolates (e.g., NK cell derived from cord blood). In some embodiments, the NK cells are from a cell line. In some embodiments, the NK cells are fresh cells. In some embodiments, the NK cells were previously frozen and thawed.

In some embodiments, the NK cells comprise a chimeric antigen receptor (CAR). The NK cell can comprise any CAR, including for example one or more of a CD19 CAR, B cell maturation antigen (BCMA) CAR, glypican-3 (GPC3) CAR, CD22 CAR, mesothelin CAR, MUC1 CAR, epithelial cell adhesion molecule (EpCAM) CAR, epidermal growth factor receptor (EGFR) CAR, CD123 CAR, CD20 CAR, HER2 CAR, GD2 CAR, CD133 CAR, EphA2 CAR, and a prostate-specific membrane antigen (PSMA) CAR. Accordingly, in some embodiments, the NK cells comprise a CD19 CAR. In some embodiments, the NK cells comprise a BCMA CAR, In some embodiments, the NK cells comprise a GPC3 CAR. In some embodiments, the NK cells comprises a CD22 CAR. In some embodiments, the NK cells comprise a mesothelin CAR. In some embodiments, the NK cells comprise a MUC1 CAR. In some embodiments, the NK cells comprise an EpCAM CAR. In some embodiments, the NK cells comprise a EGFR CAR. In some embodiments, the NK cells comprise a CD123 CAR. In some embodiments, the NK cells comprise a CD20 CAR. In some embodiments, the NK cells comprise a HER2 CAR. In some embodiments, the NK cells comprise a GD2 CAR. In some embodiments, the NK cells comprise a CD133 CAR. In some embodiments, the NK cells comprise a EphA2 CAR. In some embodiments, the NK cells comprise a PSMA CAR.

In some embodiments, the NK cells are engineered to express one or more cytokines. In some embodiments, the NK cells are engineered to express one or more of IL-15, complex of IL-15 and IL-15Ra, IL-18, IL-12, IL-7, CCL19. Accordingly, in some embodiments, the NK cells are engineered to express IL-15. In some embodiments, the NK cells are engineered to express a complex of IL-15 and IL-15Ra. In some embodiments, the NK cells are engineered to express IL-18. In some embodiments, the NK cells are engineered to express IL-12. In some embodiments, the NK cells are engineered to express IL-7. In some embodiments, the NK cells are engineered to express CCL19.

In some embodiments, the NK cells are engineered to express one or more suicide genes. For example, in some examples the NK cells are engineered to express one or more of iCaspase9, non-secretable TNFalpha, herpes simplex virus thymidine kinase (HSV-TK), Uracil phosphoribosyl transferase (UPRTase), Cytosine deaminase (CD). Accordingly, in some embodiments, the NK cells are engineered to express one or more of iCaspase9. In some embodiments, the NK cells are engineered to express non-secretable TNFalpha. In some embodiments, the NK cells are engineered to express herpes simplex virus thymidine kinase (HSV-TK). In some embodiments, the NK cells are engineered to express Uracil phosphoribosyl transferase (UPRTase). In some embodiments, the NK cells are engineered to express Cytosine deaminase (CD).

In some embodiments, the NK cells are engineered to express CD19-CAR, IL-15, and iCaspase9. An exemplary CAR-NK cell comprising CD19 IL-15, and iCaspase9 is described in Leukemia 32 (2018)520-531, incorporated herein by reference in its entirety.

In some embodiments, the genetically engineered cord blood NK cells include a CD19-CAR comprising an anti-CD19 binding domain, a transmembrane domain such as the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154 and an intracellular signaling domain such as an intracellular signaling domain FcR gamma, FcR beta, CD3 gamma, CD3 delta, CD3-zeta, CD3 epsilon, CD5, CD22, CD79a, CD79b, and CD66d. The CD-19 binding domain can be a single chain antibody or single chain antibody fragment, such as an scFv. In one embodiment, the anti-CD19 binding domain comprises a light chain variable region comprising the amino acid sequence set forth in SEQ ID NO: 1 and/or a heavy chain variable region comprising the amino acid sequence set forth in SEQ ID NO: 2. In another embodiment, the CD-19 CAR can include an anti-CD19 binding domain, a CD28 transmembrane domain (an exemplary CD28 transmembrane sequence is shown in SEQ ID NO: 3, a CD3z signaling domain (an exemplary CD3z sequence is shown in SEQ ID NO: 4 and can further include a suicide switch such as iCaspase9 and/or IL-15.

In one embodiment, the genetically engineered cord blood NK cells include a nucleic acid molecule encoding the heavy chain variable region of an anti-CD19 binding domain and/or a nucleic acid molecule encoding the light chain variable region of an anti-CD19 binding domain.

In some embodiments, a cryopreservation medium described herein comprises CAR-NK cells at a concentration of between 6 M/mL to 120 M/mL. In some embodiments, a cryopreservation medium described herein comprises CAR-NK cells at a concentration of between 6 M/mL to 200 M/mL. In some embodiments, the cryopreservation medium comprises CAR-NK cells at a concentration of between 5 M/mL to 25 M/mL. In some embodiments, a cryopreservation medium described herein comprises CAR-NK cells at a concentration of between 6 M/mL to 120 M/mL in a 36 mL volume. In some embodiments, a cryopreservation medium described herein comprises CAR-NK cells at a concentration of between 6 M/mL to 200 M/mL in a 36 mL volume. In some embodiments, the cryopreservation medium comprises CAR-NK cells at a concentration of between 5 M/mL to 25 M/mL in a 36 mL volume.

In some embodiments, the total volume of cryopreservation medium in which the CAR-NK cells are suspended is between about 15 mL and 30 mL, about 30 mL and 45 mL, about 30 and 60 mL, or about 30 mL and 75 mL. Accordingly, in some embodiments, the total volume in which the NK cells are suspended is between about 15 mL and 30 mL. In some embodiments, the total volume in which the CAR-NK cells are suspended is between about 30 mL and 45 mL. In some embodiments, the total volume in which the CAR-NK cells are suspended is between about 30 mL and 60 mL. In some embodiments, the total volume in which the CAR-NK cells are suspended is between about 30 mL and 75 mL. In some embodiments, the total volume in which the CAR-NK cells are suspended is about 20 mL, 21 mL, 22 mL, 23 mL, 24 mL, 25 mL, 26 mL, 27 mL, 28 mL, 29 mL, 30 mL, 31 mL, 32 mL, 33 mL, 34 mL, 35 mL, 36 mL, 37 mL, 38 mL, 39 mL, 40 mL, 41 mL, 42 mL, 43 mL, 44 mL, 45 mL, 46 mL, 47 mL, 48 mL, 49 mL or 50 mL. In some embodiments, the total volume in which the CAR-NK cells are suspended is about 20 mL. In some embodiments, the total volume in which the CAR-NK cells are suspended is about 21 mL. In some embodiments, the total volume in which the CAR-NK cells are suspended is about 22 mL. In some embodiments, the total volume in which the CAR-NK cells are suspended is about 23 mL. In some embodiments, the total volume in which the CAR-NK cells are suspended is about 24 mL. In some embodiments, the total volume in which the CAR-NK cells are suspended is about 25 mL. In some embodiments, the total volume in which the CAR-NK cells are suspended is about 26 mL. In some embodiments, the total volume in which the CAR-NK cells are suspended is about 27 mL. In some embodiments, the total volume in which the CAR-NK cells are suspended is about 28 mL. In some embodiments, the total volume in which the CAR-NK cells are suspended is about 29 mL. In some embodiments, the total volume in which the CAR-NK cells are suspended is about 30 mL. In some embodiments, the total volume in which the CAR-NK cells are suspended is about 31 mL. In some embodiments, the total volume in which the CAR-NK cells are suspended is about 32 mL. In some embodiments, the total volume in which the CAR-NK cells are suspended is about 33 mL. In some embodiments, the total volume in which the CAR-NK cells are suspended is about 34 mL. In some embodiments, the total volume in which the CAR-NK cells are suspended is about 35 mL. In some embodiments, the total volume in which the CAR-NK cells are suspended is about 36 mL. In some embodiments, the total volume in which the CAR-NK cells are suspended is about 37 mL. In some embodiments, the total volume in which the CAR-NK cells are suspended is about 38 mL. In some embodiments, the total volume in which the CAR-NK cells are suspended is about 39 mL. In some embodiments, the total volume in which the CAR-NK cells are suspended is about 40 mL. In some embodiments, the total volume in which the CAR-NK cells are suspended is about 41 mL. In some embodiments, the total volume in which the CAR-NK cells are suspended is about 42 mL. In some embodiments, the total volume in which the CAR-NK cells are suspended is about 43 mL. In some embodiments, the total volume in which the CAR-NK cells are suspended is about 44 mL. In some embodiments, the total volume in which the CAR-NK cells are suspended is about 45 mL. In some embodiments, the total volume in which the CAR-NK cells are suspended is about 46 mL.

In some embodiments, CAR-NK cells once thawed are at a concentration of about 200 million to 800 million cells per 36 mL. In some embodiments, the cryopreservation medium comprises CAR-NK cells at a concentration of between about 100-1000 million CAR-NK cells per a 36 mL fill volume. In some embodiments, the cryopreservation medium comprises CAR-NK cells at a concentration of between about 200-800 million cells per a 36 mL fill volume. In some embodiments, the cryopreservation medium comprises CAR-NK cells at a concentration of about 100 million cells per a 36 mL fill volume. In some embodiments, the cryopreservation medium comprises CAR-NK cells at a concentration of about 200 million cells per a 36 mL fill volume. In some embodiments, the cryopreservation medium comprises CAR-NK cells at a concentration of about 300 million cells per a 36 mL fill volume. In some embodiments, the cryopreservation medium comprises CAR-NK cells at a concentration of about 400 million cells per a 36 mL fill volume. In some embodiments, the cryopreservation medium comprises CAR-NK cells at a concentration of about 500 million cells per a 36 mL fill volume. In some embodiments, the cryopreservation medium comprises CAR-NK cells at a concentration of about 600 million cells per a 36 mL fill volume. In some embodiments, the cryopreservation medium comprises CAR-NK cells at a concentration of about 700 million cells per a 36 mL fill volume. In some embodiments, the cryopreservation medium comprises CAR-NK cells at a concentration of about 800 million cells per a 36 mL fill volume. In some embodiments, the cryopreservation medium comprises CAR-NK cells at a concentration of about 1000 million cells per a 36 mL fill volume.

In some embodiments, a CAR-NK cell therapy product is cryopreserved using a cryopreservation medium described herein. In some embodiments, the CAR-NK cell therapy product is an allogeneic cell therapy product comprised of human cord blood-derived NK cells transduced with a retroviral vector expressing iCaspase9, CD-19 CAR and IL-15.

In some embodiments, the NK cells comprise a CAR-NK cell therapy product comprising a population of cells between 1×10⁶ to 5×10⁹ formulated in a cryopreservation media described herein In some embodiments, the NK cells comprises a CAR-NK cell therapy product comprising a population of cells between 2×10⁶ to 800×10⁶ formulated in a cryopreservation media described herein. In some embodiments, the CAR-NK cell therapy product is an allogeneic cell therapy product comprised of 200×10⁶ to 800×10⁶ of human cord blood-derived NK cells transduced with a retroviral vector expressing iCaspase9, CD-19 CAR and IL-15 and formulated in 36 mLs of a cryopreservation medium containing DMSO, trehalose, PLASMA-LYTE A and HSA in a 50 mL AT vial. In some embodiments, the CAR-NK cell therapy product is an allogeneic cell therapy product comprised of 200×10⁶ to 800×10⁶ of viable human cord blood-derived NK cells transduced with a retroviral vector expressing iCaspase9, CD-19 CAR and IL-15 and formulated in 36 mLs of a cryopreservation medium containing DMSO, trehalose, PLASMA-LYTE A and HSA in a 50 mL AT vial.

### Method of Cryopreservation of NK Cells

Also provided herein are methods of cryopreserving cells, once formulated in a cryopreservation medium described herein.

Cells that can be cryopreserved using the media described herein, in general, include any mammalian cell. In some embodiments, cells that can be cryopreserved include stem cells, other progenitor cells, red and white blood cells, sperm cells, oocytes, ova, and cellular materials derived from tissues and organs. Further examples of suitable cells include pancreatic islet cells, chondrocytes, cells of neural origin, cells of hepatic origin, cells of opthalmolic origin, cells of orthopedic origin, cells from connective tissues, cells of reproductive origin, and cells of cardiac origin. In some other embodiments, cells include the erythrocyte, neutrophilic, eosinophilic, and basophilic granulocytes, lymphocytes, and platelet cells. In some embodiments, lymphocyte includes B-lymphocytes, T-lymphocytes, non-B-lymphocytes, non-T-lymphocytes, induced pluripotent cell-derived lymphocytes, and genetically modified lymphocytes. The lymphocytes further includes T cells. The progenitor cells include embryonic stem cells (ESC), hematopoietic progenitor cells (HPC), or induced pluripotent cells (iPS cells). In one embodiment, the cells that are cryopreserved are NK cells and in particular CAR-NK cells.

In general, it is known that when liquid water is cooled it undergoes a phase transition from liquid to solid at a critical temperature. The phase transition is a first-order transition, which means the water either absorbs or releases an amount of energy per volume known as the latent heat. During the phase transition the temperature of the water will remain constant as heat is added or removed and during this time the water is in a mixed-state, where some of it is in a liquid state and some is in a solid state. The temperature at which a phase transition happens can be called the critical temperature of the phase transition. When water is cooled the temperature of the water decreases until the critical temperature is reached. While cooling is still applied the temperature of the water remains constant until the latent heat has been removed from the water after which the temperature of the water, now in solid state, once again decreases. This means that there is a duration of time during which latent heat is being removed from the water. The time during which latent heat is removed in the process of freezing is the time when ice crystals may form, which is undesirable when cryopreserving samples containing biological materials such as cells.

The cryopreservation media described herein used with the cryopreservation methods described herein minimize the impact of latent heat during cryopreservation (i.e., ice formation impact), thereby resulting in a higher viability of cell sample, which has been frozen.

In some embodiments, the cryopreserved media and methods described herein are used for freezing cell therapies, e.g., either freshly isolated from an organism or harvested from a cell culture. In various embodiments described herein, the cells that are cryopreserved using the media and methods described herein are immune cells, e.g., NK cells derived from cord-blood, peripheral blood, T cells, iPS cells, leukapheresis products, mononuclear cells or spleen. In some embodiments, the NK cells comprises a chimeric antigen receptor (CAR).

In some aspects, a method of cryopreservation of NK cells includes contacting the NK cells with the cryopreservation medium provided herein. In another aspect, the method of cryopreservation of NK cells comprises the steps of a) providing natural killer (NK) cells in a cryopreservation medium comprising HSA, PLASMA-LYTE A, a disaccharide and CS10; 2), (b) cooling the cells at a controlled rate to a temperature of −80° C.; and (c) storing the cells in liquid nitrogen vapor phase, thereby cryopreserving the NK cells.

In some embodiments, cryopreservation is performed on cell suspensions of mammalian immune cells, such as NK cells. In some embodiments, the cells are cryopreserved in a container, e.g., a cryobag or a cryovial. In some embodiments, the cryopreservation method described herein is scalable to up to 30 vials or more. In some embodiments, the cryopreservation method is scalable to 30 vials, 50 vials, or 75 vials.

Various containers can be used in the methods of cryopreservation including, for example, cryovials or cryobags. Exemplary cryovials, include, for example, AT® vials or Nunc™ vials or glass vials. In some embodiments, current methods of cryopreservation are performed on mammalian immune cell suspensions in cryovials or AT® vials or any other suitable container. In some embodiments, suitable containers are those that are resistant to DMSO. In some embodiments, the cryovials, cryobags, AT® vials or Nunc™ vials, glass vials and other containers used for cryopreservation, are compatible for use with DMSO. In some embodiments, the cryovials, cryobags, AT® vials or Nunc™ vials, glass vials and other containers used for cryopreservation, are chemically unreactive with DMSO. In some embodiments, the containers used for cryopreservation are both DEHP-free and DMSO-resistant (e.g., AT® vials). In various embodiments, the containers used herein (e.g., AT® vials) facilitate aseptic transfer of cells directly into a subject in need thereof.

Containers used herein can have various dimensions, including those dimensions discussed herein. In some embodiments, the dimensions suitable for use with cryovials as discussed herein are also suitable for use with other containers, such as AT® vials or Nunc™ vials.

In some embodiments, the cryovials have a dimension between about 5 mm external diameter and 100 mm height. In some embodiments, the cryovials have a dimension of about 10 mm external diameter and 75 mm height. In some embodiments, the cryovials have a dimension, of 10 mm external diameter and 50 mm height.

In some embodiments, the cryovials have a dimension of 10 mm external diameter and 48.3 mm height. In some embodiments, the cryovials have a dimension of 10.5 mm external diameter and 48.3 mm height. In some embodiments, the cryovials have a dimension of 11.0 mm external diameter and 48.3 mm height. In some embodiments, the cryovials have a dimension of between 11.5 mm external diameter and 48.3 mm height. In some embodiments, the cryovials have a dimension of 12.0 mm external diameter and 48.3 mm height. In some embodiments, the cryovials have a dimension of 12.5 mm external diameter and 48.3 mm height. In some embodiments, the cryovials have a dimension of 13.0 mm external diameter and 48.3 mm height. In some embodiments, the cryovials have a dimension of 13.5 mm external diameter and 48.3 mm height. In some embodiments, the cryovials have a dimension of 14.0 mm external diameter and 48.3 mm height. In some embodiments, the cryovials have an external of 14.5 mm external diameter and 48.3 mm height. In some embodiments, the cryovials have a dimension of 15.0 mm external diameter and 48.3 mm height.

In some embodiments, the cryovial has a height of between about 30 mm to about 85 mm. In some embodiments, the cryovial has an external diameter of about between 15 mm to about 40 mm. In some embodiments, the cryovial has a maximum volume capacity of between about 1 mL and 55 mL. Various kinds of cryovials are suitable for the compositions and methods described herein. Exemplary cryovials, including description of cryovial dimensions can be found at http://www.aseptictech.com/sites/default/files/brochure_vialslines_v3.0.pdf the contents of which are incorporated herein by reference in its entirety.

In some embodiments, the cryovials have a height of between about 45 mm and 100 mm. In some embodiments, the cryovials have a height of 45.3 mm. In some embodiments, the cryovials have a height of 45.6 mm. In some embodiments, the cryovials have a height of 46 mm. In some embodiments, the cryovials have a height of 46.3 mm. In some embodiments, the cryovials have a height of 46.6 mm. In some embodiments, the cryovials have a height of 47 mm. In some embodiments, the cryovials have a height of 47.3 mm. In some embodiments, the cryovials have a height of 47.6 mm. In some embodiments, the cryovials have a height of 48 mm. In some embodiments, the cryovials have a height of 48.3 mm. In some embodiments, the cryovials have a height of 48.6 mm. In some embodiments, the cryovials have a height of 49 mm. In some embodiments, the cryovials have a height of 49.3 mm. In some embodiments, the cryovials have a height of 50 mm. In some embodiments, the cryovials have a height of 50 mm. In some embodiments, the cryovials have a height of 50 mm. In some embodiments, the cryovials have a height of 55 mm. In some embodiments, the cryovials have a height of 60 mm. In some embodiments, the cryovials have a height of 55 mm. In some embodiments, the cryovials have a height of 65. In some embodiments, the cryovials have a height of 55 mm. In some embodiments, the cryovials have a height of 70 mm. In some embodiments, the cryovials have a height of 75 mm. In some embodiments, the cryovials have a height of 80 mm. In some embodiments, the cryovials have a height of 85 mm. In some embodiments, the cryovials have a height of 90 mm. In some embodiments, the cryovials have a height of 95 mm. In some embodiments, the cryovials have a height of 100 mm.

In some embodiments, the cryovials have an external diameter of 10 mm. In some embodiments, the cryovials have an external diameter of 10.5 mm. In some embodiments, the cryovials have an external diameter of 11 mm. In some embodiments, the cryovials have an external diameter of 11.5 mm. In some embodiments, the cryovials have an external diameter of 12 mm. In some embodiments, the cryovials have an external diameter of 12.5 mm. In some embodiments, the cryovials have an external diameter of 13 mm. In some embodiments, the cryovials have an external diameter of 13.5 mm. In some embodiments, the cryovials have an external diameter of 14 mm. In some embodiments, the cryovials have an external diameter of 14.5 mm. In some embodiments, the cryovials have an external diameter of 15 mm.

In some embodiments, the cryobags have a width of 11 cm. In some embodiments, the cryobags have a width of 11.3 cm. In some embodiments, the cryobags have a width of 11.5 cm. In some embodiments, the cryobags have a width of 11.7 cm. In some embodiments, the cryobags have a width of 11.9 cm. In some embodiments, the cryobags have a width of 12.1 cm. In some embodiments, the cryobags have a width of 12.3 cm. In some embodiments, the cryobags have a width of 12.5 cm. In some embodiments, the cryobags have a width of 12.7 cm. In some embodiments, the cryobags have a width of 12.9 cm. In some embodiments, the cryobags have a width of 13.1 cm. In some embodiments, the cryobags have a width of 13.3 cm. In some embodiments, the cryobags have a width of 13.5 cm. In some embodiments, the cryobags have a width of 13.7 cm.

In some embodiments, the cryobags have a length of 14.1 cm. In some embodiments, the cryobags have a length of 14.3 cm. In some embodiments, the cryobags have a length of 14.5 cm. In some embodiments, the cryobags have a length of 14.7 cm. In some embodiments, the cryobags have a length of 14.9 cm. In some embodiments, the cryobags have a length of 15.1 cm. In some embodiments, the cryobags have a length of 15.3 cm. In some embodiments, the cryobags have a length of 15.5 cm. In some embodiments, the cryobags have a length of 15.7 cm. In some embodiments, the cryobags have a length of 15.9 cm. In some embodiments, the cryobags have a length of 16.1 cm. In some embodiments, the cryobags have a length of 16.3 cm. In some embodiments, the cryobags have a length of 16.5 cm. In some embodiments, the cryobags have a length of 16.7 cm.

In some embodiments the volume of the cryovials (i.e., maximum capacity) can be between 2 mL to 50 mL for example, 2 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, 20 mL, 21 mL, 22 mL, 23 mL, 24 mL, 25 mL, 26 mL, 27 mL, 28 mL, 29 mL, 30 mL, 31 mL, 32 mL, 33 mL, 34 mL, 35 mL, 36 mL, 37 mL, 38 mL, 39 mL, 40 mL, 41 mL, 42 mL, 43 mL, 44 mL, 45 mL, 46 mL, 47 mL, 48 mL, 49 mL, 50 mL, 51 mL, 52 mL, 53 mL, 54 mL, 55 mL, 56 mL, 57 mL, 58 mL, 59 mL, 60 mL, 61 mL, 62 mL, 63 mL, 64 mL, 65 mL, 66 mL, 67 mL, 68 mL, 69 mL, 70 mL, 71 mL, 72 mL, 73 mL, 74 mL, 75 mL, 76 mL, 77 mL, 78 mL, 79 mL, 80 mL, 81 mL, 82 mL, 83 mL, 84 mL, 85 mL, 86 mL, 87 mL, 88 mL, 89 mL, 90 mL, 91 mL, 92 mL, 93 mL, 94 mL, 95 mL, 96 mL, 97 mL, 98 mL, 99 mL, or 100 mL.

As used herein, “fill volume” refers to the volume of the sample comprising cells in a container. In some embodiments, the fill volume is less than the maximum capacity of the container. In some embodiments, the fill volume in vials can be between 15% to 90% maximum capacity of the vial. For example, the fill volume in vials can be 15% maximum capacity, 20% maximum capacity, 25% maximum capacity, 30% maximum capacity, 35% maximum capacity, 40% maximum capacity, 45% maximum capacity, 50% maximum capacity, 55% maximum capacity, 60% maximum capacity, 65% maximum capacity, 70% maximum capacity, 75% maximum capacity, 80% maximum capacity, 85% maximum capacity or 90% maximum capacity. In some embodiments, a 2 mL cryovial has a fill volume of 1 mL. In some embodiments, a 50 mL cryovial has a fill volume of 8 mL to 45 mL. In some embodiments, a 50 mL cryovial has a fill volume of 8 mL. In some embodiments, a 50 mL cryovial has a fill volume of 10 mL. In some embodiments, a 50 mL cryovial has a fill volume of 12 mL. In some embodiments, a 50 mL cryovial has a fill volume of 14 mL. In some embodiments, a 50 mL cryovial has a fill volume of 16 mL. In some embodiments, a 50 mL cryovial has a fill volume of 18 mL. In some embodiments, a 50 mL cryovial has a fill volume of 20 mL. In some embodiments, a 50 mL cryovial has a fill volume of 22 mL. In some embodiments, a 50 mL cryovial has a fill volume of 24 mL. In some embodiments, a 50 mL cryovial has a fill volume of 26 mL. In some embodiments, a 50 mL cryovial has a fill volume of 28 mL. In some embodiments, a 50 mL cryovial has a fill volume of 30 mL. In some embodiments, a 50 mL cryovial has a fill volume of 32 mL. In some embodiments, a 50 mL cryovial has a fill volume of 34 mL. In some embodiments, a 50 mL cyrovial has a fill volume of 36 mL. In some embodiments, a 50 mL cyrovial has a fill volume of 38 mL. In some embodiments, a 50 mL AT vial has a fill volume of 40 mL. In some embodiments, a 50 mL cyrovial has a fill volume of 42 mL. In some embodiments, a 50 mL cyrovial has a fill volume of 44 mL. In some embodiments, a 50 mL cyrovial has a fill volume of 45 mL. In some embodiments, the cryogenic container can have a volume of about 50 mL, about 75 mL, about 100 mL, about 250 mL, about 500 mL, about 750 mL, about 1 L, or more than about 1 L.

In some embodiments, the mammalian immune cells (e.g., NK cells and in particular CAR-NK cells) can be cryopreserved at a concentration between about 6 M/mL to about 120 M/mL. For example, in some embodiments, immune cells can be cryopreserved at a concentration of 6 M/mL. In some embodiments, immune cells can be cryopreserved at a concentration of 10 M/mL. In some embodiments, immune cells can be cryopreserved at a concentration of 15 M/mL. In some embodiments, immune cells can be cryopreserved at a concentration of 20 M/mL. In some embodiments, immune cells can be cryopreserved at a concentration of 25 M/mL. In some embodiments, immune cells can be cryopreserved at a concentration of 30 M/mL. In some embodiments, immune cells can be cryopreserved at a concentration of 35 M/mL. In some embodiments, immune cells can be cryopreserved at a concentration of 40 M/mL. In some embodiments, immune cells can be cryopreserved at a concentration of 45 M/mL. In some embodiments, immune cells can be cryopreserved at a concentration of 50 M/mL. In some embodiments, immune cells can be cryopreserved at a concentration of 55 M/mL. In some embodiments, immune cells can be cryopreserved at a concentration of 60 M/mL. In some embodiments, immune cells can be cryopreserved at a concentration of 65 M/mL. In some embodiments, immune cells can be cryopreserved at a concentration of 70 M/mL. In some embodiments, immune cells can be cryopreserved at a concentration of 75 M/mL. In some embodiments, immune cells can be cryopreserved at a concentration of 80 M/mL. In some embodiments, immune cells can be cryopreserved at a concentration of 85 M/mL. In some embodiments, immune cells can be cryopreserved at a concentration of 90 M/mL. In some embodiments, immune cells can be cryopreserved at a concentration of 100 M/mL. In some embodiments, immune cells can be cryopreserved at a concentration of 105 M/mL. In some embodiments, immune cells can be cryopreserved at a concentration of 110 M/mL. In some embodiments, immune cells can be cryopreserved at a concentration of 115 M/mL. In some embodiments, immune cells can be cryopreserved at a concentration of 120 M/mL. In some embodiments, immune cells can be cryopreserved at a concentration of 130 M/mL. In some embodiments, immune cells can be cryopreserved at a concentration of 140 M/mL. In some embodiments, immune cells can be cryopreserved at a concentration of 150 M/mL. In some embodiments, immune cells can be cryopreserved at a concentration of 160 M/mL. In some embodiments, immune cells can be cryopreserved at a concentration of 170 M/mL. In some embodiments, immune cells can be cryopreserved at a concentration of 180 M/mL. In some embodiments, immune cells can be cryopreserved at a concentration of 190 M/mL. In some embodiments, immune cells can be cryopreserved at a concentration of 200 M/mL.

It is to be understood, that in some embodiments, the thawed CAR NK cells are at a quantity of between about 100 million and 1000 million viable cells. In some embodiments, the CAR NK cells are provided in a dosage of about between 1×10⁶ to 1×10⁹ cells in a volume of about 36 mL in a 50 mL cryovial (e.g., an AT vial). In some embodiments, the CAR NK cells are provided in a dosage of about between 200×10⁶ and 800×10⁶ cells in a 50 mL cryovial (e.g., an AT vial). In some embodiments, the CAR NK cells are suspended in a cryopreservation medium as described herein, followed by cryopreservation as disclosed herein. Such cryopreserved NK cells can then be stored as described herein. The sample comprising cryopreserved NK cells are then thawed as described herein. The thawed cells are subsequently administered to a patient in need thereof. In some embodiments, a volume of about 33 mL, 34 mL, 35 mL, or 36 mL of the thawed cells are administered to a patient in need thereof using a vial adapter for aseptic transfer for administration. In some embodiments, a volume of about 33 mL of the thawed cells are administered to a patient in need thereof using a vial adapter for aseptic transfer to a syringe for administration. In some embodiments, a volume of about 34 mL of the thawed cells are administered to a patient in need thereof using a vial adapter for aseptic transfer to a syringe administration. In some embodiments, a volume of about 35 mL of the thawed cells are administered to a patient in need thereof using a vial adapter for aseptic transfer to a syringe for administration. In some embodiments, a volume of about 36 mL of the thawed cells are administered to a patient in need thereof using a vial adapter for aseptic transfer to a syringe for administration.

In some aspects, a method of cryopreservation of NK cells includes (a) providing NK cells (e.g., CAR-NK cells) in a cryopreservation medium comprising a cryoprotectant, an albumin, a disaccharide, and a non-pyrogenic and isotonic crystalloid solution; (b) cooling the cells to a temperature of −80° C.; and (c) storing the cells in liquid nitrogen vapor phase, thereby cryopreserving the NK cells. In some aspects, a method of cryopreservation of NK includes (a) providing a container comprising a sample comprising NK cells suspended in a cryopreservation medium, wherein the volume of the sample is at least 5% less than the maximum capacity of the container, and wherein the sample volume is at least 10 mL; (b) cooling the container from a temperature above freezing temperature of the sample to a temperature of about or below −80° C. in a multi-step process at a controlled rate to minimize latent heat of fusion; and (c) storing the cells in liquid nitrogen vapor phase, thereby cryopreserving the immune cells.

In some embodiments, a method of cryopreserving a sample comprising cells suspended in a media described herein comprises changing temperature of the sample from a first temperature to a final temperature of less than or equal to −80° C., thereby cryopreserving the sample at the final temperature. In some embodiments, such a method comprises the steps of (a) placing the sample at a first temperature above the freezing temperature of the sample; (b) reducing the first temperature to a second temperature at a first controlled rate, where the second temperature is at least 2° C. lower than the first temperature; (c) reducing the second temperature to a third temperature at a second controlled rate, where the third temperature is at least 40° C. lower than the second temperature; (d) increasing the third temperature to a fourth temperature at a third controlled rate, where the fourth temperature is at least 20° C. higher than the third temperature; (e) reducing the fourth temperature to a fifth temperature at a fourth controlled rate, where the fifth temperature is at least 10° C. lower than the fourth temperature; and (f) reducing the fifth temperature to the final temperature at a fifth controlled rate, where the final temperature is less than or equal to −80° C.

In some embodiments, the first temperature is about 4° C. to 0° C.

In some embodiments, the first controlled rate is between about 0.75° C. and 1.25° C. per minute.

In some embodiments, the second temperature is about −2° C.

In some embodiments, the second controlled rate is between about 20° C. and 30° C. per minute.

In some embodiments, the third temperature is about −60° C.

In some embodiments, the third controlled rate is between about 5° C. and 15° C. per minute.

In some embodiments, the fourth temperature is about −25° C.

In some embodiments, the fourth controlled rate is between 0.5° C. and 1.25° C. per minute.

In some embodiments, the fifth temperature is about −40° C.

In some embodiments, the fifth controlled rate is between 7° C. and 15° C. per minute.

In some embodiments, final temperature is less than or equal to −80° C.

In some aspects, a method is provided comprising cryopreserving engineered immune cells (e.g., CAR-NK cells or CAR-T cells) suitable for cell therapy using a cryopreservation media described herein, the method comprising stepwise freezing a population of engineered immune cells at a controlled rate to minimize latent heat of fusion, where the stepwise freezing comprises cooling the cells at a rate of between 0.5° C. per minute to 30° C. per minute to a final temp of −80° C. or below, thereby cryopreserving the cells.

In some aspects, a method of thawing the cryopreserved engineered immune cells is provided, the method comprising heating a container comprising the cryopreserved engineered immune cells (e.g., NK cells or CAR-NK cells) to a temperature of between 37° C. and 70° C.; and agitating the cells at a speed of between about 100 and about 250 RPM for a suitable period of time until the cells are thawed.

In some embodiments, the agitating the cells is at a speed of about between 100 RPM to about 250 RPM. In some embodiments, the agitating the cells is at a speed of about between 100 RPM to about 150 RPM. In some embodiments, the agitating the cells is at a speed of about between 100 RPM to about 125 RPM. In some embodiments, the agitating the cells is at a speed of about 100 RPM. In some embodiments, the agitating the cells is at a speed of about 125 RPM. In some embodiments, the agitating the cells is at a speed of about 150 RPM. In some embodiments, the agitating the cells is at a speed of about 200 RPM. In some embodiments, the agitating the cells is at a speed of about 250 RPM.

In some embodiments, the total time of thawing is about between 5 minutes and 20 minutes. Accordingly, in some embodiments, the total time of thawing is about 5 minutes. In some embodiments, the total time of thawing is about 10 minutes. In some embodiments, the total time of thawing is about 15 minutes. In some embodiments, the total time of thawing is about 20 minutes.

In some embodiments, the total time of thawing is about between 5 minutes and 20 minutes for a 50 mL vial. Accordingly, in some embodiments, the total time of thawing is about 5 minutes for a 50 mL vial. In some embodiments, the total time of thawing is about 10 minutes for a 50 mL vial. In some embodiments, the total time of thawing is about 15 minutes for a 50 mL vial. In some embodiments, the total time of thawing is about 20 minutes for a 50 mL vial.

In some embodiments, the thawed cells are stable for between about 1 to 6 hours. Accordingly, in some embodiments, the thawed cells are stable for about between 2 to 4 hours. In some embodiments, the thawed cells are stable for about between 1 to 2 hours. In some embodiments, the thawed cells are stable for about 1 hour. In some embodiments, the thawed cells are sable for about 2 hours. In some embodiments, the thawed cells are stable for about 3 hours. In some embodiments, the thawed cells are stable for about 4 hours. In some embodiments, the thawed cells are stable for about 5 hours. In some embodiments, the thawed cells are stable for more than 5 hours.

In some embodiments, cells suitable for adoptive cell therapy (e.g., CAR-NK cells, CAR-T cells and the like) are cryopreserved in a media described herein using a cryopreservation method described herein. Such cells may be cryopreserved at one location in a cryovial at a cell dose suitable for single administration to a patient and then shipped at a temperature of about −140° C. to a different location (e.g., point of care location) where the cells are thawed using a thawing process described herein and administered to a patient in need thereof using a vial adapter.

Accordingly, also provided herein are methods of transporting mammalian cells cryopreserved in media described herein, the method comprising: (a) providing mammalian cells in a cryopreservation medium at a first location; (b) cooling the cells to −80° C. at the first location at a controlled rate to minimize latent heat of fusion in accordance with the disclosures herein, thereby cryopreserving the mammalian cells; and (c) transporting the cryopreserved mammalian cells to a second location (e.g., point of care location) at a temperature of between about −20° C. to about −140° C. or below.

In various embodiments, cells may be thawed at the second location (e.g., point of care location) prior to administration to a subject in need thereof.

The first location may coincide with the location where the cells are frozen or cryopreserved and the second location may coincide with the location where a subject in need thereof is present. It is understood that the first and second location may be present in the same geographic location or be geographically separated by a certain distance, e.g., a few miles or a different state, country or continent.

In some embodiments, the genetically modified immune cells used in adoptive cell therapy (e.g., CAR-NK cells) once thawed have cell survival similar to that of fresh cells isolated from the same donor. In some embodiments, the genetically modified immune cells used in adoptive cell therapy (e.g., CAR-NK cells), once thawed, have cell survival in vitro similar to that of fresh cells isolated from the same donor. In some embodiments, the genetically modified immune cells used in adoptive cell therapy (e.g., CAR-NK cells), once thawed, have similar cell survival following transplantation into a subject in comparison to fresh cells isolated from the same donor. In some embodiments, the genetically modified immune cells used in adoptive cell therapy (e.g., CAR-NK cells), once thawed, maintain cellular activity and function similar to a freshly isolated NK cell that has not been frozen, or that has been once frozen and subsequently thawed. In some embodiments, the genetically modified immune cells are suitable for therapeutic use.

In some embodiments, the cryopreservation medium is maintained at room temperature prior to use and the NK cells are held at room temperature for as long as thirty minutes before freezing is commenced without an appreciable loss in cell viability. In some other embodiments, the cryopreservation medium is cooled to about 4° C. prior to the addition of NK cells in order to expedite the freezing process, and requires that the freezing process commence immediately after the cryopreservation medium is added to the cells in order to minimize cell death induced by the presence of solution.

In some embodiments, cells are suspended in the cryopreservation medium to prepare a cell suspension, the suspension thus prepared is dispensed into freezing tubes, and the resulting tubes are placed directly in an ultra-low temperature freezer at −80° C. or below to freeze the cells. In some other embodiments, the freezing tubes can be placed in a programmed freezer to freeze the cells at a controlled rate. The preservation of the frozen cells can be carried out by maintaining the cells at the temperature used for freezing (for example, −80° C.). There are multiple forms of computerized freezing chambers that reduce the temperature a degree at a time until it reaches −80° C.; they often include a computerized strategy of having the cells linger for somewhat longer at a temperature at which ice begins to form to minimize ice crystal damage to the cells.

In some embodiments, freezing comprises: (a) cooling cells from an initial temperature to a final temperature of about −80° C. using solid carbon dioxide, or (b) cooling cells from an initial temperature to a final temperature of between about −140° C. to about −196° C. using liquid nitrogen.

In some embodiments, an optimal freezing method comprises: (a) providing cells in a cryopreservation medium such as that described herein; (b) cooling the cells to −80° C. at a controlled rate to minimize latent heat of fusion; and (c) storing the cells in liquid nitrogen vapor phase, thereby cryopreserving the cells. In some embodiments, the controlled rate to minimize latent heat of fusion comprises one or more steps of cooling the cells at a rate of between 0.75° C. per minute to 30° C. per minute to a final temperature of −80° C. or below. In some embodiments, total time for achieving cryopreservation of the cells is one hour or less.

In some embodiments, the optimal freezing program for CAR-NK cells comprises the following steps: (a) placing the cells at 4° C.; (b) reducing the temperature at a rate of between about 0.75° C. and 1.25° C. per minute to a temperature of about −2.0° C.; (c) maintaining the cells at about −2.0° C. for between about 2-4 minutes; (d) reducing the temperature at a rate of between about 20° C. and 30° C. per minute to a temperature of about −60° C.; (e) maintaining the cells at about −60° C. for between about 30 seconds and 2 minutes; (f) increasing the temperature at a rate of between about 5° C. and 15° C. per minute to a temperature of about −25° C.; (g) maintaining the cells at about −25° C. for about between 5 minutes and 15 minutes; (h) reducing temperature by at a rate of between 0.75° C. and 1.25° C. per minute to a temperature of about −40° C.; (i) maintaining the cells at about −40° C. for between about 2 and 7 minutes; (j) and reducing the temperature at a rate of between about 7° C. and 15° C. per minute to a temperature of −80° C.

In some embodiments, the thawed cells using the medium and/or methods described herein have a viability of at least about 70%, 75%, 80%, 85%, 90%, 95%, 99% or more. The thawed cells are viable for between about 1 and 5 hours. In some embodiments, the thawed cells are viable for about 1 hour. In some embodiments, the thawed cells are viable for about 2 hours. In some embodiments, the thawed cells are viable for about 3 hours. In some embodiments, the thawed cells are viable for about 4 hours. In some embodiments, the thawed cells are viable for about 5 hours.

In some embodiments, the container holding the cells is stable at cryogenic temperatures and allow for rapid heat transfer for effective control of both freezing and thawing. Sealed plastic vials (e.g., Nunc and Wheaton cryules) or glass ampules can be used for multiple small amounts (1 to 2 mL), while larger volumes of 100 to 200 mL can be frozen in polyolefin bags, such as those available from Fenwal, held between metal plates. Other exemplary containers for cryopreserving cells include cryovials and/or cryobags. Exemplary cryovials, include for example AT vials.

In some embodiments, the frozen cells are then transferred to a long-term cryogenic storage vessel. In some embodiments, samples are cryogenically stored in liquid nitrogen (−196° C.) or liquid nitrogen vapor (−105° C.). Such storage is greatly facilitated by the availability of highly efficient liquid nitrogen refrigerators. In some embodiments, the frozen cells are shipped at a temperature of about −140° C. or below. In some embodiments, the frozen cells are shipped at a temperature of between about −140° C. and −196° C. In some embodiments, the cells are frozen and stored at −140° C., −196° C. or below and subsequently shipped at −140° C., −196° C.

When the cells are needed, the frozen cells and the composition are subjected to a thawing process, after which the cells can be recovered. In some embodiments, the thawing process is rapid and is scalable to up to 5 vials. In some embodiments, the thawing process is rapid and is scalable to up to 10 vials, 15 vials, 20 vials, 25 vials, 30 vials or more.

In some embodiments, thawing the cells comprises: (a) heating a water bath to a temperature ranging from 37° C. and 70° C.; (b) transferring a container comprising cryopreserved immune cells into the pre-heated water bath; and agitating the container at a speed of between about 100 and about 250 RPM for a suitable period of time, thereby to obtain thawed immune cells.

In some embodiments, thawing the cells comprises: (a) heating a dry-thawing device to a temperature ranging from 37° C. and 70° C.; (b) transferring a container comprising cryopreserved immune cells into the a pre-heated dry-thawing device; and agitating the container at a speed of between about 100 and about 250 RPM for a suitable period of time, thereby to obtain thawed immune cells.

In some embodiments, thawing can be accomplished in a water bath or in a dry-thawing device which can uniformly distribute heat throughout the cryopreserved samples to thaw the sample.

In some embodiments, the thawing is accomplished by adjusting the temperature of the water bath to 40° C. In some embodiments, the thawing is accomplished by adjusting the temperature of the water bath to 45° C. In some embodiments, the thawing is accomplished by adjusting the temperature of the water bath to 50° C. In some embodiments, the thawing is accomplished by adjusting the temperature of the water bath to 55° C. In some embodiments, the thawing is accomplished by adjusting the temperature of the water bath to 60° C. In some embodiments, the thawing is accomplished by adjusting the temperature of the water bath to 65° C. In some embodiments, the thawing is accomplished by adjusting the temperature of the water bath to 70° C.

In some embodiments, the thawing is accomplished by using dry heat, such as that produced by a dry-thawing device. Accordingly, in some embodiments, the thawing is accomplished by adjusting the temperature of a dry-thawing device to 40° C. In some embodiments, the thawing is accomplished by adjusting the temperature of a dry-thawing device to 45° C. In some embodiments, the thawing is accomplished by adjusting the temperature of a dry-thawing device to 50° C. In some embodiments, the thawing is accomplished by adjusting the temperature of a dry-thawing device to 55° C. In some embodiments, the thawing is accomplished by adjusting the temperature of a dry-thawing device to 60° C. In some embodiments, the thawing is accomplished by adjusting the temperature of a dry-thawing device to 65° C. In some embodiments, the thawing is accomplished by adjusting the temperature of a dry-thawing device to 70° C.

In some embodiments, the thawing is accomplished in combination with agitation of the sample.

In some embodiments, the thawing is accomplished by adjusting the rotational speed of an orbital shaker water bath to between 100 rpm and 250 rpm. In some embodiments, the thawing is accomplished by adjusting the rotational speed of the orbital shaker water bath to 120 rpm. In some embodiments, the thawing is accomplished by adjusting the rotational speed of the orbital shaker water bath to 125 rpm. In some embodiments, the thawing is accomplished by adjusting the rotational speed of the orbital shaker water bath to 130 rpm. In some embodiments, the thawing is accomplished by adjusting the rotational speed of the orbital shaker water bath to 135 rpm. In some embodiments, the thawing is accomplished by adjusting the rotational speed of the orbital shaker water bath to 140 rpm. In some embodiments, the thawing is accomplished by adjusting the rotational speed of the orbital shaker water bath to 145 rpm. In some embodiments, the thawing is accomplished by adjusting the rotational speed of the orbital shaker water bath to 150 rpm. In some embodiments, the thawing is accomplished by adjusting the rotational speed of the orbital shaker water bath more than 150 rpm.

In some embodiments, the thawing is accomplished by adjusting the rotational speed of a sample in a dry heating device to between 100 rpm and 250 rpm. In some embodiments, the thawing is accomplished by adjusting the rotational speed of a sample in a dry heating device to 120 rpm. In some embodiments, the thawing is accomplished by adjusting the rotational speed of a sample in a dry heating device to 125 rpm. In some embodiments the thawing is accomplished by adjusting the rotational speed of a sample in a dry heating device to 130 rpm. In some embodiments, the thawing is accomplished by adjusting the rotational speed of a sample in a dry heating device to 135 rpm. In some embodiments, the thawing is accomplished by adjusting the rotational speed of a sample in a dry heating device to 140 rpm. In some embodiments, the thawing is accomplished by adjusting the rotational speed of a sample in a dry heating device to 145 rpm. In some embodiments, the thawing is accomplished by adjusting the rotational speed of a sample in a dry heating device to 150 rpm. In some embodiments, the thawing is accomplished by adjusting the rotational speed of a sample in a dry heating device to more than 150 rpm.

In some embodiments, the speed of agitation does not cause shearing of cells being thawed.

In some embodiments, the thawing is accomplished by adjusting the temperature of the orbital shaker water bath to 37° C. and the rotational speed to 100 rpm. In some embodiments, the thawing is accomplished by adjusting the temperature of the orbital shaker water bath to 37° C. and the rotational speed to 125 rpm. In some embodiments, the thawing is accomplished by adjusting the temperature of the orbital shaker water bath to 37° C. and the rotational speed to 150 rpm. In some embodiments, the thawing is accomplished by adjusting the temperature of the orbital shaker water bath to 37° C. and the rotational speed to 175 rpm. In some embodiments, the thawing is accomplished by adjusting the temperature of the orbital shaker water bath to 37° C. and the rotational speed to 200 rpm. In some embodiments, the thawing is accomplished by adjusting the temperature of the orbital shaker water bath to 45° C. and the rotational speed to 100 rpm. In some embodiments, the thawing is accomplished by adjusting the temperature of the orbital shaker water bath to 45° C. and the rotational speed to 125 rpm. In some embodiments, the thawing is accomplished by adjusting the temperature of the orbital shaker water bath to 45° C. and the rotational speed to 150 rpm. In some embodiments, the thawing is accomplished by adjusting the temperature of the orbital shaker water bath to 45° C. and the rotational speed to 175 rpm. In some embodiments, the thawing is accomplished by adjusting the temperature of the orbital shaker water bath to 45° C. and the rotational speed to 200 rpm. In some embodiments, the thawing is accomplished by adjusting the temperature of the orbital shaker water bath to 50° C. and the rotational speed to 100 rpm. In some embodiments, the thawing is accomplished by adjusting the temperature of the orbital shaker water bath to 50° C. and the rotational speed to 125 rpm. In some embodiments, the thawing is accomplished by adjusting the temperature of the orbital shaker water bath to 50° C. and the rotational speed to 150 rpm. In some embodiments, the thawing is accomplished by adjusting the temperature of the orbital shaker water bath to 50° C. and the rotational speed to 175 rpm. In some embodiments, the thawing is accomplished by adjusting the temperature of the orbital shaker water bath to 50° C. and the rotational speed to 200 rpm. In some embodiments, the thawing is accomplished by adjusting the temperature of the orbital shaker water bath to 60° C. and the rotational speed to 100 rpm. In some embodiments, the thawing is accomplished by adjusting the temperature of the orbital shaker water bath to 60° C. and the rotational speed to 125 rpm. In some embodiments, the thawing is accomplished by adjusting the temperature of the orbital shaker water bath to 60° C. and the rotational speed to 150 rpm. In some embodiments, the thawing is accomplished by adjusting the temperature of the orbital shaker water bath to 60° C. and the rotational speed to 175 rpm. In some embodiments, the thawing is accomplished by adjusting the temperature of the orbital shaker water bath to 60° C. and the rotational speed to 200 rpm.

In some embodiments, the thawing is accomplished by adjusting the temperature of the dry heating device to 37° C. and the rotational speed to 100 rpm. In some embodiments, the thawing is accomplished by adjusting the temperature of the dry heating device to 37° C. and the rotational speed to 125 rpm. In some embodiments, the thawing is accomplished by adjusting the temperature of the dry heating device to 37° C. and the rotational speed to 150 rpm. In some embodiments, the thawing is accomplished by adjusting the temperature of the dry heating device to 37° C. and the rotational speed to 175 rpm. In some embodiments, the thawing is accomplished by adjusting the temperature of the dry heating device to 37° C. and the rotational speed to 200 rpm. In some embodiments, the thawing is accomplished by adjusting the temperature of the dry heating device to 45° C. and the rotational speed to 100 rpm. In some embodiments, the thawing is accomplished by adjusting the temperature of the dry heating device to 45° C. and the rotational speed to 125 rpm. In some embodiments, the thawing is accomplished by adjusting the temperature of the dry heating device to 45° C. and the rotational speed to 150 rpm. In some embodiments, the thawing is accomplished by adjusting the temperature of the dry heating device to 45° C. and the rotational speed to 175 rpm. In some embodiments, the thawing is accomplished by adjusting the temperature of the dry heating device to 45° C. and the rotational speed to 200 rpm. In some embodiments, the thawing is accomplished by adjusting the temperature of the dry heating device to 50° C. and the rotational speed to 100 rpm. In some embodiments, the thawing is accomplished by adjusting the temperature of the dry heating device to 50° C. and the rotational speed to 125 rpm. In some embodiments, the thawing is accomplished by adjusting the temperature of the dry heating device to 50° C. and the rotational speed to 150 rpm. In some embodiments, the thawing is accomplished by adjusting the temperature of the dry heating device to 50° C. and the rotational speed to 175 rpm. In some embodiments, the thawing is accomplished by adjusting the temperature of the dry heating device to 50° C. and the rotational speed to 200 rpm. In some embodiments, the thawing is accomplished by adjusting the temperature of the dry heating device to 60° C. and the rotational speed to 100 rpm. In some embodiments the thawing is accomplished by adjusting the temperature of the dry heating device to 60° C. and the rotational speed to 125 rpm. In some embodiments, the thawing is accomplished by adjusting the temperature of the dry heating device to 60° C. and the rotational speed to 150 rpm. In some embodiments, the thawing is accomplished by adjusting the temperature of the dry heating device to 60° C. and the rotational speed to 175 rpm. In some embodiments the thawing is accomplished by adjusting the temperature of the dry heating device to 60° C. and the rotational speed to 200 rpm. In some embodiments, the thawing is accomplished by adjusting the temperature of the dry heating device to 70° C. and the rotational speed to 100 rpm. In some embodiments the thawing is accomplished by adjusting the temperature of the dry heating device to 70° C. and the rotational speed to 125 rpm. In some embodiments, the thawing is accomplished by adjusting the temperature of the dry heating device to 70° C. and the rotational speed to 150 rpm. In some embodiments, the thawing is accomplished by adjusting the temperature of the dry heating device to 70° C. and the rotational speed to 175 rpm. In some embodiments the thawing is accomplished by adjusting the temperature of the dry heating device to 70° C. and the rotational speed to 200 rpm. In some embodiments, the thawing is accomplished by adjusting the temperature of the dry heating device to 80° C. and the rotational speed to 100 rpm. In some embodiments the thawing is accomplished by adjusting the temperature of the dry heating device to 80° C. and the rotational speed to 125 rpm. In some embodiments, the thawing is accomplished by adjusting the temperature of the dry heating device to 80° C. and the rotational speed to 150 rpm. In some embodiments, the thawing is accomplished by adjusting the temperature of the dry heating device to 80° C. and the rotational speed to 175 rpm. In some embodiments the thawing is accomplished by adjusting the temperature of the dry heating device to 80° C. and the rotational speed to 200 rpm. In some embodiments, the thawing is accomplished by adjusting the temperature of the dry heating device to 90° C. and the rotational speed to 100 rpm. In some embodiments the thawing is accomplished by adjusting the temperature of the dry heating device to 90° C. and the rotational speed to 125 rpm. In some embodiments, the thawing is accomplished by adjusting the temperature of the dry heating device to 90° C. and the rotational speed to 150 rpm. In some embodiments, the thawing is accomplished by adjusting the temperature of the dry heating device to 90° C. and the rotational speed to 175 rpm. In some embodiments the thawing is accomplished by adjusting the temperature of the dry heating device to 90° C. and the rotational speed to 200 rpm.

In some embodiments, the thawing is accomplished by incubating the cryopreserved cell suspension in the orbital shaker water bath for about 5 min. In some embodiments, the thawing is accomplished by incubating the cryopreserved cell suspension in the orbital shaker water bath for about 6 min. In some embodiments, the thawing is accomplished by incubating the cryopreserved cell suspension in the orbital shaker water bath for about 7 min. In some embodiments, the thawing is accomplished by incubating the cryopreserved cell suspension in the orbital shaker water bath for about 8 min. In some embodiments, the thawing is accomplished by incubating the cryopreserved cell suspension in the orbital shaker water bath for about 9 min. In some embodiments, the thawing is accomplished by incubating the cryopreserved cell suspension in the orbital shaker water bath for about 10 min. In some embodiments, the thawing is accomplished by incubating the cryopreserved cell suspension in the orbital shaker water bath for about 11 min. In some embodiments, the thawing is accomplished by incubating the cryopreserved cell suspension in the orbital shaker water bath for about 12 min. In some embodiments, the thawing is accomplished by incubating the cryopreserved cell suspension in the orbital shaker water bath for about 13 min. In some embodiments, the thawing is accomplished by incubating the cryopreserved cell suspension in the orbital shaker water bath for about 14 min. In some embodiments, the thawing is accomplished by incubating the cryopreserved cell suspension in the orbital shaker water bath for about 15 min.

In some embodiments, the thawing is accomplished by incubating the cryopreserved cell suspension in the dry heating device for about 5 min. In some embodiments, the thawing is accomplished by incubating the cryopreserved cell suspension in the dry heating device for about 6 min. In some embodiments, the thawing is accomplished by incubating the cryopreserved cell suspension in the dry heating device for about 7 min. In some embodiments, the thawing is accomplished by incubating the cryopreserved cell suspension in the dry heating device for about 8 min. In some embodiments, the thawing is accomplished by incubating the cryopreserved cell suspension in the dry heating device for about 9 min. In some embodiments, the thawing is accomplished by incubating the cryopreserved cell suspension in dry heating device for about 10 min. In some embodiments, the thawing is accomplished by incubating the cryopreserved cell suspension in the dry heating device for about 11 min. In some embodiments, the thawing is accomplished by incubating the cryopreserved cell suspension in dry heating device for about 12 min. In some embodiments, the thawing is accomplished by incubating the cryopreserved cell suspension in the dry heating device for about 13 min. In some embodiments, the thawing is accomplished by incubating the cryopreserved cell suspension in the dry heating device for about 14 min. In some embodiments, the thawing is accomplished by incubating the cryopreserved cell suspension in the dry heating device bath for about 15 min.
Thawing cells in this manner, such as for example CAR-NK cells, allows for thawed cells to retain high viability (e.g., greater than 70%, 75%, 80%, 85%, 90%, 95%, or greater than 95%) and functionality similar to cells that have not been cryopreserved following CAR transduction.

The thawed cells can be used for a variety of applications as described further below.

### Uses of Cryopreserved Cells

The compositions described herein, including the CAR-NK cell compositions contained within the cryopreservation medium described herein, are suitable for adoptive cell therapy. Adoptive cell therapies can be used to treat various disease, including, for example, cancer. In certain embodiments, the CAR-NK cell compositions contained within the cryopreservation medium described herein is useful for the treatment of a cancer or a tumor. In certain embodiments, the cancer comprises breast, heart, lung, small intestine, colon, spleen, kidney, bladder, head, neck, ovarian, prostate, brain, pancreatic, skin, bone, bone marrow, blood, thymus, uterine, testicular, and liver tumors. In some embodiments, the cancer is a blood cancer. In some embodiments, the blood cancer is a B-cell malignancy (e.g., diffuse large B-cell lymphoma).

In some embodiments, the cryopreservation media described herein is used to suspend cells used for adoptive cell therapy. Accordingly, in some embodiment, CAR-NK cell compositions are suspended in the cryopreservation media described herein.

In some embodiments, the CAR-NK cell compositions suspended in the cryopreservation media described herein is used to treat a subject who has cancer. In some embodiments, the subject is administered a composition comprising CAR-NK cells within the cryopreservation media described herein. In some embodiments, the CAR-NK cell comprises an anti-CD19 CAR gene and an IL-15 gene. In some embodiments, the CAR-NK cell comprises an anti-CD19 CAR gene, an IL-15 gene, and iCaspase9. In some embodiments, the CAR-NK cells are not washed prior to administering to a subject in need thereof. In some embodiments, the CAR-NK cells are washed of the cryopreservation media prior to administering to a subject in need thereof. In some embodiments, the thawed cells are administered into a patient in need thereof within about 30 minutes and 2 hours from thawing the cells. In some embodiments, the rate of intravenous infusion into a subject is between about 2-3 minutes.

In some embodiments, the adoptive cell therapy is used in combination with one or more additional cancer treatments, such as for example lymphodepleting chemotherapy. Accordingly, in some embodiments, a subject who has cancer receives lymphodepleting chemotherapy before administration of a CAR-NK cell therapy product formulated in a cryopreservation media described herein.

In some embodiments, a CAR-NK cell therapy product is cryopreserved as described herein and subsequently thawed prior to administration to a patient in need thereof. For example, a CAR-NK cell therapy product as described herein is cryopreserved, transported, thawed, and administered to a patient in need thereof as described herein. Accordingly, in some embodiments, the CAR-NK cell therapy product is cryopreserved in a formulation as described herein and subsequently thawed prior to administration to a patient for treatment of B-cell malignancies. In some embodiments, a CAR-NK cell therapy product cryopreserved as described herein and subsequently thawed prior to administration to a patient in need thereof is a CAR-NK cell therapy product comprising a CD19-CAR comprising an anti-CD19 binding domain, a transmembrane domain such as the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154 and an intracellular signaling domain such as an intracellular signaling domain FcR gamma, FcR beta, CD3 gamma, CD3 delta, CD3-zeta, CD3 epsilon, CD5, CD22, CD79a, CD79b, and CD66d. The CD-19 binding domain can be a single chain antibody or single chain antibody fragment, such as an scFv. In one embodiment, the anti-CD19 binding domain comprises a light chain variable region comprising the amino acid sequence set forth in SEQ ID NO: 1 and/or a heavy chain variable region comprising the amino acid sequence set forth in SEQ ID NO: 2. In another embodiment, the CD-19 CAR can include an anti-CD19 binding domain, a CD28 transmembrane domain (an exemplary CD28 transmembrane sequence is shown in SEQ ID NO: 3, a CD3z signaling domain (an exemplary CD3z sequence is shown in SEQ ID NO: 4 and can further include a suicide switch such as iCaspase9 and/or IL-15.

In one embodiment, the CAR-NK cell therapy product comprises a nucleic acid molecule encoding the heavy chain variable region of an anti-CD19 binding domain and/or a nucleic acid molecule encoding the light chain variable region of an anti-CD19 binding domain.

In some embodiments, the frozen CAR-NK cell therapy product is frozen in a vial (e.g., a 50 mL AT vial) in a cryopreservation media described herein and using a method described herein and transported or shipped in the same vial at a temperature ranging from −140° C. to −196° C. to a location where a patient is situated, such that the cells can be thawed at the location where the patient is situated and administered aseptically directly to the patient using a syringe connected to the vial with a vial adapter (i.e., vial to vein transfer). For example, in some embodiments, a method of transporting the cell therapy product comprises: (a) providing the CAR-NK cells in a cryopreservation medium as described herein; (b) cooling the CAR-NK cells to a temperature of −80° C., thereby cryopreserving the mammalian cells; and (c) transporting the cryopreserved mammalian cells to a different location at a temperature of between about −20° C. to about −140° C. or below. Accordingly, in some embodiments, the cryopreserved mammalian cells are transported to a different location in a container maintained at a temperature at or below −140° C. In some embodiments, the cryopreserved mammalian cells are transported to a different location in a container maintained at a temperature of between −140° C. and −196° C. In some embodiments, the cryopreserved mammalian cells are transported to a different location in a cryoshipper. In some embodiments, the transported cells may be stored in a cryoshipper until administration to a patient. In some embodiments, the transported cells are stored at the different location at a temperature at or below −140° C. In some embodiments, the transported cells are stored at the different location at a temperature of between −140° C. to −196° C. In some embodiments, the transported cells are stored in liquid nitrogen vapor phase from the time of receipt at the different location until a future time of use. The storage, thus can be accomplished using clinical site freezers or tanks that maintain temperature at or below −140° C. in a vapor phase of liquid nitrogen. In some embodiments, the cell therapy product is a population of CD19-CAR NK cells that further comprise IL-15 and iCaspase9. In some embodiments, the cell therapy product is cryopreserved in a container at concentration of between about 6 and 120 million cells per milliliter. In some embodiments, the cell therapy product is cryopreserved in a 50 mL container at concentration of between about 6 and 120 million cells per milliliter. In some embodiments, the cell therapy product is cryopreserved in a 50 mL container at concentration of between about 3 and 150 million cells per milliliter. In some embodiments, the cell therapy product is cryopreserved in a 50 mL container at concentration of between about 1 and 250 million cells per milliliter. In some embodiments, the cell therapy product is cryopreserved in a 50 mL container at concentration of between about 1 and 350 million cells per milliliter. In some embodiments, the cell therapy product is cryopreserved in a 50 mL container at a concentration of between about 1 and 500 million cells per milliliter.

In some embodiments, the cell therapy product comprises between about 20×10⁶ and 100×10⁷ cells in 50 mL container. In some embodiments, the cell therapy product comprises between about 100×10⁶ and 900×10⁶ cells in 50 mL container. In some embodiments, the cell therapy product comprises about 50×10⁶ cells in 50 mL container. In some embodiments, the cell therapy product comprises about 100×10⁶ cells in 50 mL container. In some embodiments, the cell therapy product comprises about 200×10⁶ cells in 50 mL container. In some embodiments, the cell therapy product comprises about 200×10⁶ cells in 50 mL container. In some embodiments, the cell therapy product comprises about 300×10⁶ cells in 50 mL container. In some embodiments, the cell therapy product comprises about 400×10⁶ cells in 50 mL container. In some embodiments, the cell therapy product comprises about 500×10⁶ cells in 50 mL container. In some embodiments, the cell therapy product comprises about 600×10⁶ cells in 50 mL container. In some embodiments, the cell therapy product comprises about 700×10⁶ cells in 50 mL container. In some embodiments, the cell therapy product comprises about 800×10⁶ cells in 50 mL container. In some embodiments, the cell therapy product comprises about 900×10⁶ cells in 50 mL container. In some embodiments, the cell therapy product comprises about 1000×10⁶ cells in 50 mL container. In some embodiments, the cell therapy product comprises about 1500×10⁶ cells in 50 mL container. In some embodiments, the cell therapy product comprises about 2000×10⁶ cells in 50 mL container. In some embodiments, the cell therapy product comprises about 2500×10⁶ cells in 50 mL container. In some embodiments, the cell therapy product comprises about 3000×10⁶ cells in 50 mL container. In some embodiments, the cell therapy product comprises about 3500×10⁶ cells in 50 mL container. In some embodiments, the cell therapy product comprises about 4000×10⁶ cells in 50 mL container. In some embodiments, the cell therapy product comprises about 4500×10⁶ cells in 50 mL container. In some embodiments, the cell therapy product comprises about 5000×10⁶ cells in 50 mL container.

In some embodiments, the cell therapy product is contained in a 50 mL container at a fill volume of about between 20-45 mL. In some embodiments, the cell therapy product is contained in a 50 mL container at a fill volume of about 36 mL. In some embodiments, the cell therapy product is an immune cell, such as an NK cell, T cell or B cell. In some embodiments, the immune cell is engineered to comprise one or more transgenes, for example a chimeric antigen receptor (CAR). In some embodiments, the cells are CAR-NK+ cells. In some embodiments, the cell therapy product comprises a CD19-CAR, IL-15 transgene and an iCaspase9. In some embodiments, the cell therapy product comprises between about 100×10⁶ and 900×10⁶ CAR-NK+ cells in a 50-mL container. In some embodiments, the cell therapy product present is 200×10⁶ CAR-NK+ cells in a 50-mL container. In some embodiments, the cell therapy product present is 300×10⁶ CAR-NK+ cells in a 50-mL container. In some embodiments, the cell therapy product present is 400×10⁶ CAR-NK+ cells in a 50-mL container. In some embodiments, the cell therapy product present is 500×10⁶ CAR-NK+ cells in a 50-mL container. In some embodiments, the cell therapy product present is 600×10⁶ CAR-NK+ cells in a 50-mL container. In some embodiments, the cell therapy product present is 700×10⁶ CAR-NK+ cells in a 50-mL container. In some embodiments, the cell therapy product present is 800×10⁶ CAR-NK+ cells in a 50-mL container. In some embodiments, the cell therapy product present is 900×10⁶ CAR-NK+ cells in a 50-mL container.

The transported cell therapy product can be thawed as described herein followed by administration to a patient in need thereof. In some embodiments, the cell therapy product is thawed at the patient's bedside. In some embodiments, the cell therapy product is not washed prior to administration into a patient in need thereof.

In some embodiments, the transported cell therapy product remains frozen for further storage at the different location. In some embodiments, the thawed cells is introduced into a subject in need thereof without separating the cells and the cryopreservation solution. Thus, in some embodiments, the thawed cells are not washed prior to use. The thawed cells and accompanying cryopreservation solution is preferably warmed to body temperature (i.e., about 37° C.) prior to introduction into the subject. In such situation, the dose of the cells is based on the pre-freeze cell count.

In some embodiments, thawed cells are further cultured. In some embodiments, culturing involves placing the cells in an incubator; removing the buffer solution; and replacing the buffer solution with a culture medium designed for the growth and/or differentiation of cells. In some embodiments, the cells are incubated in the incubator for between about 6 to 7 hours. In some embodiments, the culture medium designed for the growth and/or differentiation of cells comprises Kubota's medium and/or a hormonally defined medium (HDM) for the differentiation of cells.

Viability of thawed cells can be assessed in vitro using various methods known in the art. In some embodiments, the in vitro cell viability tests includes the Trypan Blue exclusion assay. In some embodiments, other analytical methods can be used to assess the cell viability of thawed cells that had been frozen with the different cryopreservation medium, for example, flow cytometry based viability markers and the like. A person of ordinary skill in the art can opt for any analytical method to assess the viability of thawed cells that can be applied to assess the cell viability of otherwise fresh cells.

Phenotype and function of thawed cells can be assessed in vitro using various methods known in the art. In some embodiments, the in vitro cell phenotyping tests includes flow cytometry assays. In some embodiments, the in vitro cell function test includes cytokine production, cytotoxicity, proliferation and other analytical methods.

Efficacy of thawed cells in vivo can be assessed using animal studies known in the art. In some embodiments, the in vitro cell phenotyping tests immunodeficiency mice based tumor models.

The cryopreserved and thawed cells using the cryopreservation media described herein allows for using the cells for any purpose that a primary cell or fresh cell isolate can have. The cryopreserved and thawed cells retain high viability (e.g., greater than 70%, 75%, 80%, 85%, 90%, 95%, or greater than 95%) and retain physiological characteristics of their native state, which allows the cells to be used for a variety of applications, such as for genetic manipulation of the cells, and for cell therapy purposes such as, for example, in adoptive cell therapy applications.

### Sequences Disclosed Herein:

Anti-CD19 Light chain variable fragment, VL:
(SEQ ID NO: 1)
DIQMTQTTSSLSASLGDRVTISCRASQDISKYLNWYQQKPDGTVKLLIY
HTSRLHSGVPSRFSGSGSGTDYSLTISNLEQEDIATYFCQQGNTLPYTF
GGGTKLELKR
Anti-CD19 Heavy chain variable fragment, VH:
(SEQ ID NO: 2)
EVQLQQSGPGLVAPSQSLSVTCTVSGVSLPDYGVSWIRQPPRKGLEWLG
VIWGSETTYYNSALKSRLTIIKDNSKSQVFLKMNSLQTDDTAIYYCAKH
YYYGGSYAMDYWGQGTTVTVSSYVTVSSQDPA
CD28:
(SEQ ID NO: 3)
FWVLVVVGGVLACYSLLVTVAFIIFWVRSKRSRLLHSDYMNMTPRRPGP
TRKHYQPYAPPRDFAAYRS
CD32:
(SEQ ID NO: 4)
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPRGP

### EXAMPLES

Other features, objects, and advantages of the present invention are apparent in the examples that follow. It should be understood, however, that the examples, while indicating embodiments of the present invention, are given by way of illustration only, not limitation. Various changes and modifications within the scope of the invention will become apparent to those skilled in the art from the examples.

CAR-NK cells used were that comprised CD19 CAR, IL-15, and iCaspase9. It is expected that the formulations described and exemplified below will work with other adoptive cell therapy products including other CAR-NK cells. Exemplary CAR-NK cells used in these examples were genetically engineered cord blood NK cells including a CD19-CAR comprising an anti-CD19 binding domain comprising a light chain variable region comprising the amino acid sequence set forth in SEQ ID NO: 1 and/or a heavy chain variable region comprising the amino acid sequence set forth in SEQ ID NO: 2.

### Example 1: Preparation of Cryopreservation Media

This example illustrates different cryopreservation media that were prepared to cryopreserve Chimeric Antigen Receptor-Natural Killer (CAR-NK) cells.

In this example, five (5) different cryopreservation media were prepared. The cryopreservation media were cryopreservation media #1, cryopreservation media #2, cryopreservation media #3, cryopreservation media #4, and cryopreservation media #5. The composition of all these cryopreservation media are shown in Table 1. The efficacy of these cryopreservation media were tested by cryopreserving CAR-NK cells, and then evaluating their in vivo efficacy in NOD.Cg-Prkdcscid Il2rgtm1Wjl/SzJ mice (“NSG mice”).

Fresh CAR-NK cells were used as controls. As for CAR-NK cell, the frozen cord blood unit-derived NK cells that transduced gene encoding a tumor targeting CD19 CAR (iC9/CAR. 19/IL15, Leukemia 32 (2018)520-531, incorporated herein by reference in its entirety) were used. An exemplary CD19 CAR used herein is described in Leukemia 32 (2018)520-531, incorporated herein by reference in its entirety. The cryopreserved cells were thawed and injected into the NSG mice with Raji tumor cells in the following example 2.

TABLE 1
Cryopreservation Media Compositions

| Cryopreservation Media | Composition |
|---|---|
| 1 | 50% PLASMA-LYTE-HEPES + 35% |
| | Dextran/Dextrose + 10% HSA + 5% DMSO |
| 2 | 40% PLASMA-LYTE A + 50% CS10 + |
| | 10% HSA |
| 3 | 50% MEM-HEPES + 35% Dextran/Dextrose + |
| | 10% HSA + 5% DMSO |
| 4 | 40% MEM + 50% CS10 + 10% HSA |
| 5 | 38.6% PLASMA-LYTE A + 50% |
| | CS10 + 10% HSA + 0.8% AA + |
| | Vitamin (0.2% vial 1 and 0.4% |
| | vials 2) + 30 mM trehalose |

### Example 2: Comparison of the In Vivo Efficacies of Cord Blood Derived CAR NK Cells that were Preserved Using Different Cryopreservation Media

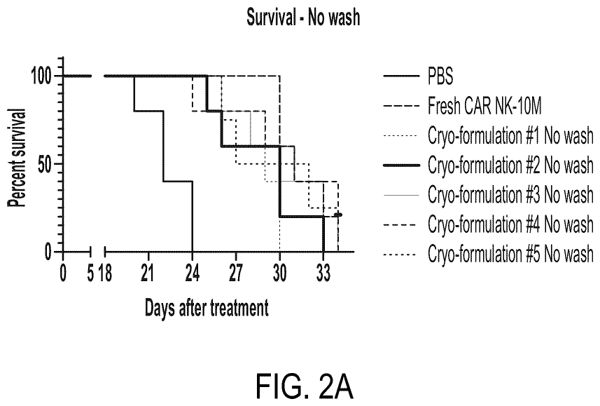

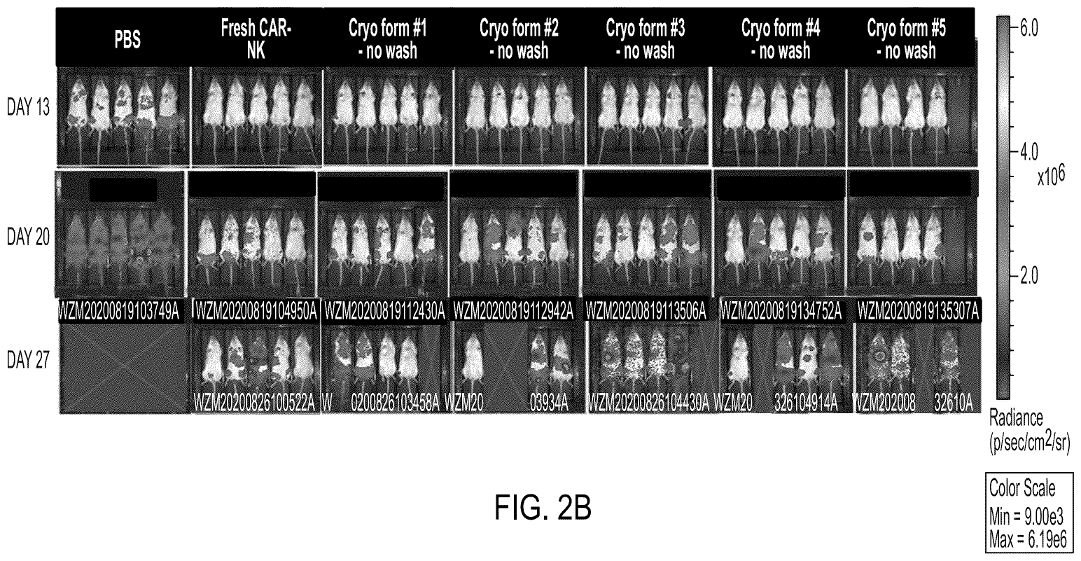

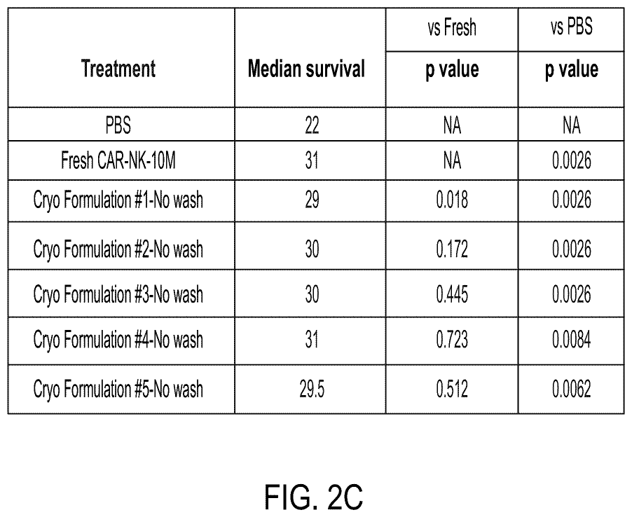

This example compares the in vivo efficacy of CAR-NK cells that were cryopreserved using 5 different cryopreservation media shown in Table 1 without washing for direct injection with PBS buffer and fresh CAR-NK cells as negative and positive control groups, respectively. Different treatment groups including 5 cryopreservation media 10M dose cells are shown in FIG. 2A-FIG. 2C. The data show that the tested formulations demonstrated efficacy similar to fresh NK cells.

The in vivo efficacy of CAR-NK cells were tested in female NOD SCID Gamma (NSG) mice that were co-administrated with luciferase expressing Raji human burkitt's lymphoma (Raji B.luc). One day before treatment (D-1) female NSG mice were randomized into groups with each group of 5 mice according to body weight and then received 1.5 Gray (Gy) of whole body irradiation. The NSG mice, female, 12-week-old were sourced from The Jackson Laboratory. On Day 0, mice were co-administrated with 2×10⁴ bioluminescent Raji B luc tumor cells and treatment via intravenous injection via tail vein. In vivo, luciferin was administered to the mice and whole body ventral images were captured nine minutes after substrate injection. Luciferase activity was measured in live mice using IVIS® Spectrum CT imaging system (PerkinElmer) in terms of total radiance flux for over 36 days on weekly basis post treatment. On the day of imaging, mice received luciferin substrate (150 mg/kg total; IP) injection and were placed in anesthesia induction chamber (2.5-3.5% isoflurane in oxygen). Upon sedation, mice were positioned in the imaging chamber for image acquisition nine minutes onwards post luciferin substrate injection. The same procedure described here will also apply to other examples where the in vivo efficacy of cells is discussed.

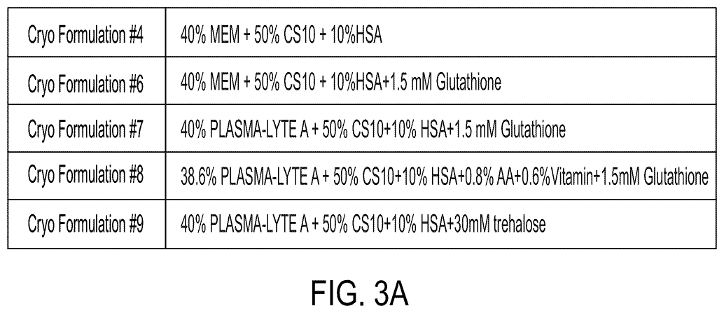

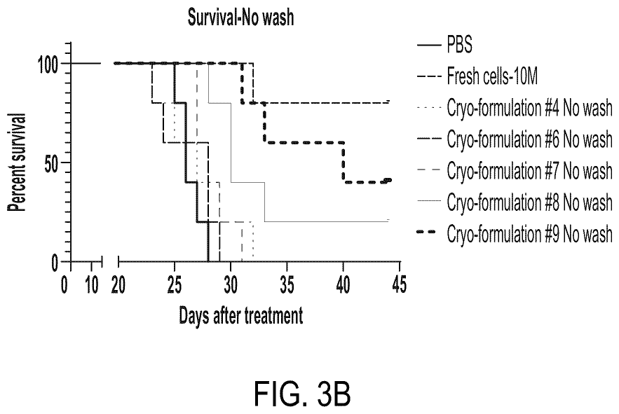

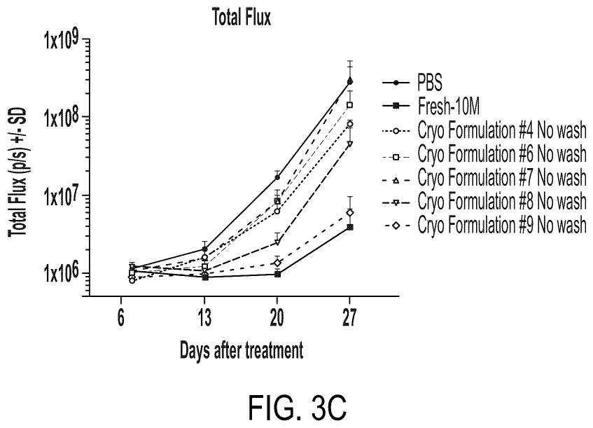

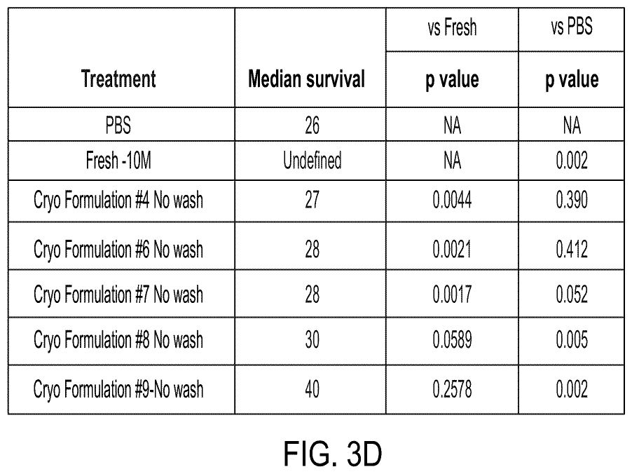

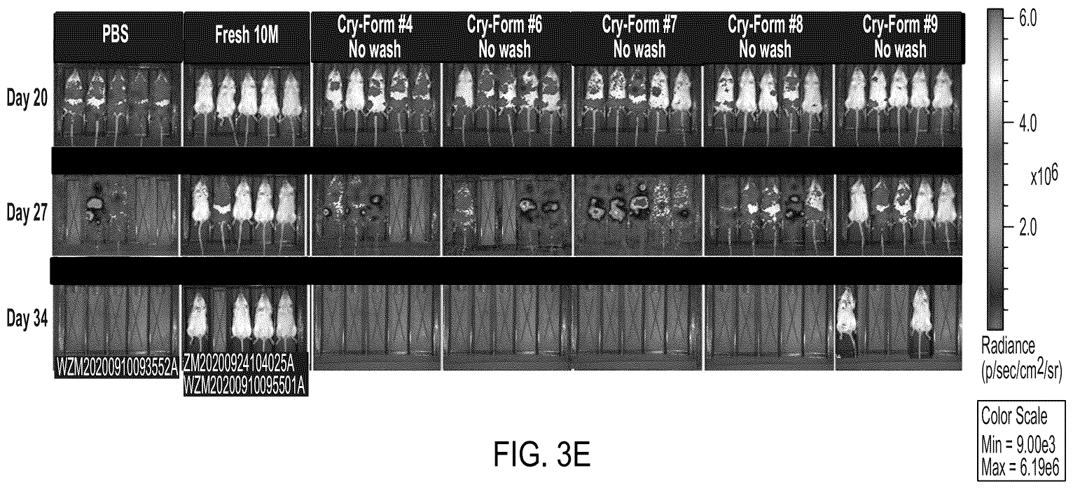

Additional cryopreservation media in vivo studies were performed. FIG. 3A provide a summary of the various cryopreservation media tested in these studies. These experiments indicated that cryopreservation media (#9, 50% PLASMA-LYTE A containing 20% HSA and 30 mM trehalose+50% CS10) had the most pronounced tumor reduction and best survival, comparable to fresh cells, indicating in vivo efficacy at reducing the amounts of Raji cells in the test animal (FIG. 3B-FIG. 3E).

Any formulation comprising suitable components for cryopreservation media described herein is suitable for the methods described. For example, a cryopreservation media comprising one or more of sodium chloride, sodium gluconate, sodium acetate trihydrate, potassium chloride, magnesium chloride, adenosine, dextran, lactobionic acid, HEPEs, sodium hydroxide, L-glutathione (reduced form), potassium chloride, potassium bicarbonate; potassium phosphate, dextrose; sucrose, mannitol, calcium chloride and magnesium chloride; sodium hydroxide, potassium hydroxide, DMSO. As a further example, suitable cryopreservation media may contain human serum albumin (HSA), Na⁺, K⁺, Mg²⁺, HEPES, one or more disaccharides, a sugar alcohol, dextran, a metabolite, and an anti-oxidant.

FIG. 3E illustrates the in vivo efficacy of CAR-NK cells on D20, D27 and D34 post-administration of CAR-NK cells. On D20 post treatment, the mice that only received PBS buffer and no CAR-NK cells showed a very high luciferase expression. The mice that received fresh CAR-NK cells only showed negligible sign of luciferase expression. The mice treated with different formulations showed a different degree of sign of luciferase expression demonstrating the in vivo efficacy of CAR-NK cells that were cryopreserved using different cryopreservation media.

FIG. 3E further illustrates the in vivo efficacy of CAR-NK cells 27 days post-administration of CAR-NK cells. The mice that only received PBS buffer and no CAR-NK cells showed even more intense luciferase expression compared to that of D20 and 3 out of 5 mice died. The mice that received CAR-NK cells in different cryopreservation media shown in FIG. 3E demonstrated different degree of signs of luciferase expression and some mice started to die. The luciferase express severity and death rate of mice was dependent on the cryopreservation media. For the cryopreservation media #9, the luciferase expression was significantly better than other cryopreservation media ion groups but similar to the group dosed with fresh cells without any mice death. On day 34, mice in PBS group and other cryopreserved CAR-NK groups died. However, 2 out of 5 mice dosed with cryopreservation media #9 survived with negligible sign of luciferase expression, close to the group dosed with fresh cells. In summary, mice treated with different formulations showed different degree of luciferase expression and survival rate demonstrating the in vivo efficacy of CAR-NK cells that were cryopreserved using different cryopreservation media. Furthermore the NK-Cells cryopreserved with cryopreservation media #9 demonstrated similar in vivo efficacy as fresh cells, significantly superior than other cryopreservation media. Table 2 lists exemplary formulation components that were developed and tested to arrive at the cryoformulations described herein.

TABLE 2
Exemplary Formulation Components Tested

| Components | Vendor |
|---|---|
| PLASMA-LYTE A | Baxter |
| MEM | Thermo Fisher |
| 25% HAS | Shire |
| Glutathione | Sigma Aldrich |
| Amino acid solution | Baxter |
| DMSO | Origen Biomedical |
| Vitamin solution | Baxter |
| CryoStor ® CS10 | BioLife Solutions |
| Trehalose | J. T. Baker |

In some embodiments, select components for the cryopreservation media include, for example, one or more of sodium chloride, sodium gluconate, sodium acetate trihydrate, potassium chloride, magnesium chloride, adenosine, dextran, lactobionic acid, HEPES, sodium hydroxide, L-glutathione (reduced form), potassium chloride, potassium bicarbonate; potassium phosphate, dextrose; sucrose, mannitol, calcium chloride and magnesium chloride; sodium hydroxide, potassium hydroxide, DMSO, human serum albumin (HSA), Na⁺, K⁺, Mg²⁺, HEPES, one or more disaccharides, a sugar alcohol, dextran, a metabolite, and an anti-oxidant.

### Example 3: In Vivo Efficacy of Rescued Fresh CAR-NK Cells Versus Cryopreserved Cells in 3 More Different Donors

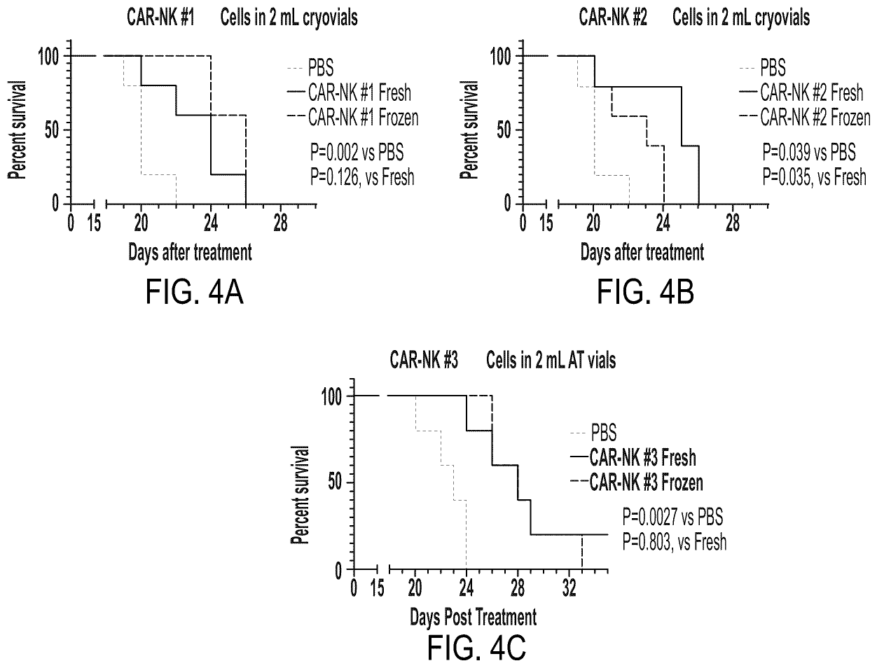

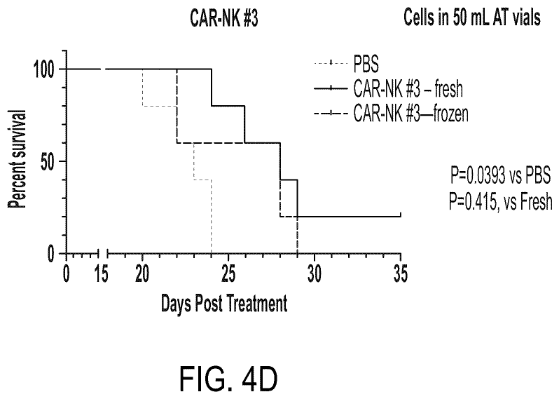

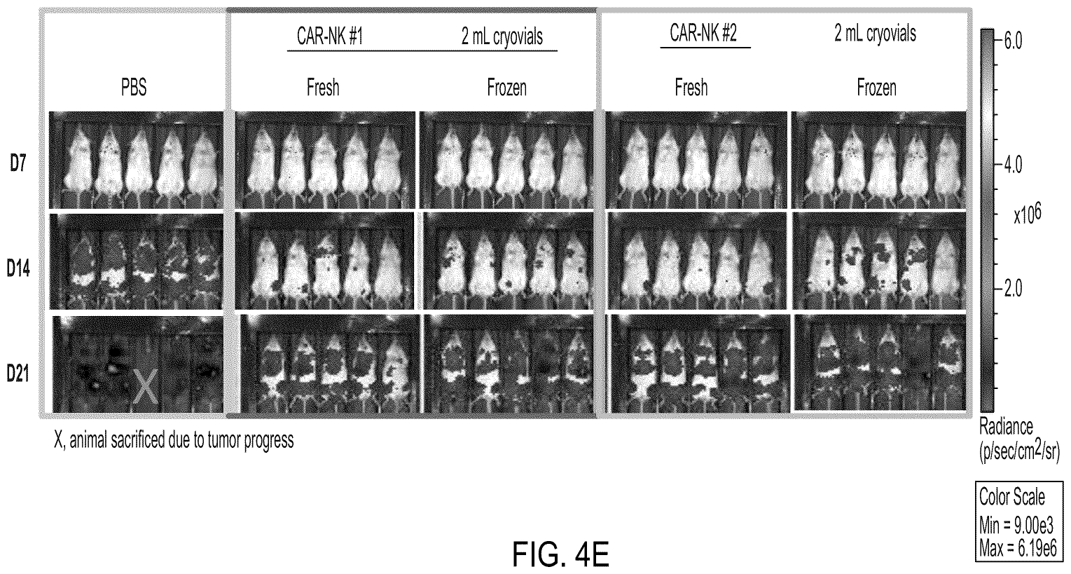

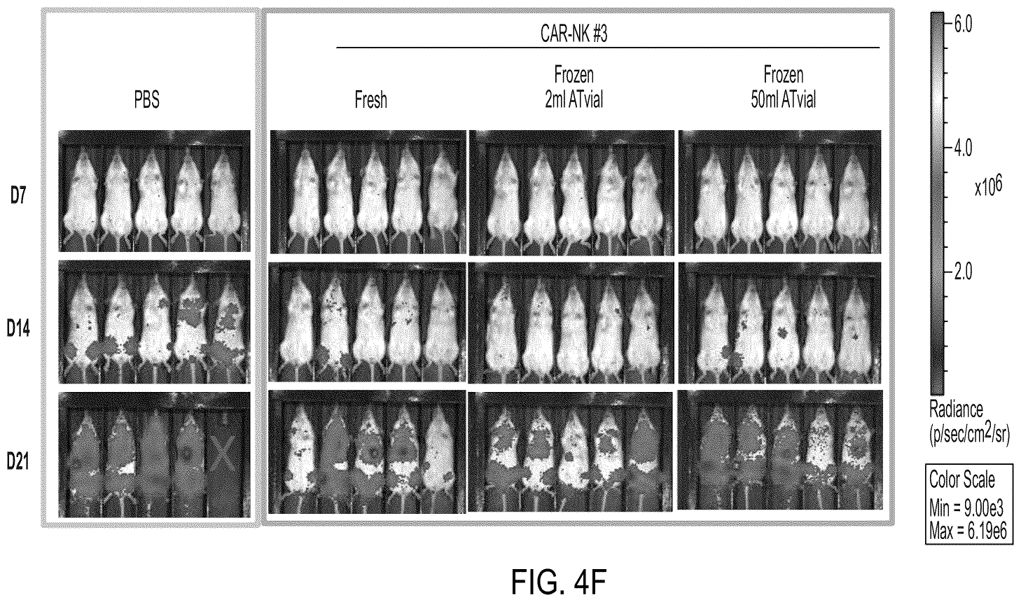

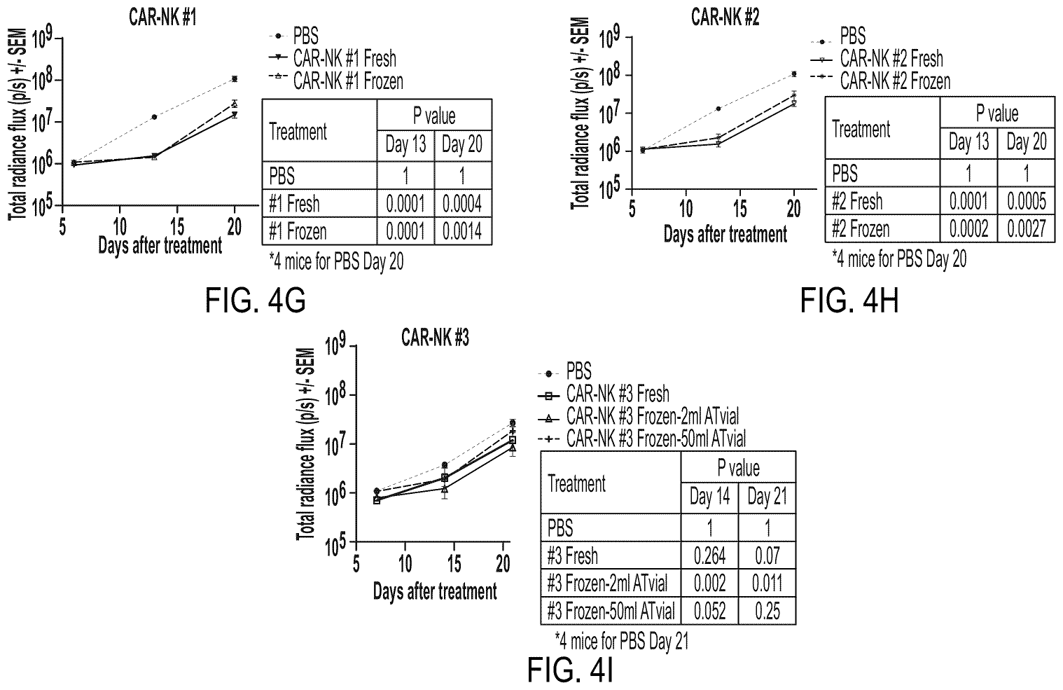

This example compares the in vivo efficacy of fresh cells vs cryopreserved CAR-NK cells. In this example, the cryoformulation #9 cryopreserved NK-cells were derived from 3 different donors. Furthermore, NK-cells cryopreserved in cryoformulation #9 was filled in different containers with different volume with different freezing and thawing procedure as provided. The results from these studies are shown in FIG. 4A-FIG. 4I.

As for CAR-NK cells, the frozen cord blood unit-derived NK cells that transduced gene encoding a tumor targeting CD19-CAR (iC9/CAR.19/IL15, Leukemia 32 (2018)520-531, incorporated herein by reference in its entirety) were used as well. Post-harvest of the CAR-NK cells and prior to the formulating, the harvested cells were washed with cold (2° C. to 8° C.) PLASMA-LYTE A containing 5-20% HSA and then centrifuged to pellet the cells. The pelleted cells were formulated with volume ratio of 1:2:1 (PLASMA-LYTE A containing 20% HSA: CS10: trehalose in PLASMA-LYTE A containing 20% HSA) and then filled in different containers, 1 mL filled in 2 mL cryovials, 1 mL filled into 2 mL closed vials and −36 mL filled into 50 mL closed vials. The CAR-NK cells were frozen using their optimal freezing program and then stored in liquid nitrogen vapor phase. The optimal freezing program for CAR-NK cells comprises the following steps: (a) placing the cells at 4° C.; (b) reducing the temperature at a rate of between about 0.75° C. and 1.25° C. per minute to a temperature of about −2.0° C.; (c) maintaining the cells at about −2.0° C. for between about 2-4 minutes; (d) reducing the temperature at a rate of between about 20° C. and 30° C. per minute to a temperature of about −60° C.; (e) maintaining the cells at about −60° C. for between about 30 seconds and 2 minutes; (f) increasing the temperature at a rate of between about 5° C. and 15° C. per minute to a temperature of about −25° C.; (g) maintaining the cells at about −25° C. for about between 5 minutes and 15 minutes; (h) reducing temperature by at a rate of between 0.75° C. and 1.25° C. per minute to a temperature of about −40° C.; (i) maintaining the cells at about −40° C. for between about 2 and 7 minutes; (j) and reducing the temperature at a rate of between about 7° C. and 15° C. per minute to a temperature of −80° C.

FIG. 4A, FIG. 4B, FIG. 4C and FIG. 4D illustrates the in vivo efficacy of CAR-NK cells after 36 days post-administration of fresh or cryopreserved NK-cells in 2 mL cryovials, 2 mL AT vials and 50 mL AT vials. The mice that only received PBS buffer (control) started to die before day 21 as shown by a cross (x) sign in FIG. 4E and FIG. 4F. The mice that received fresh and cryopreserved CAR-NK cells showed comparable luciferase expression demonstrating the in vivo efficacy of cryopreserved CAR-NK cells in cryoformulation 9 in all three donors frozen in three different containers and thawed with their individual optimized program.

FIG. 4G, FIG. 4H and FIG. 4I illustrates the in vivo efficacy of CAR-NK cells of fresh or cryopreserved CAR-NK cells. The in vivo efficacy of CAR-NK cells have been expressed in terms of total flux, a quantitative expression of the luciferase expression level. The cryopreserved CAR-NK cells in all 3 donors showed in vivo efficacy of CAR-NK cells on day 20.

### Example 4: In Vitro Efficacy and Phenotyping of Rescued Fresh CAR-NK Cells Vs Cryopreserved Cells in Different Donors

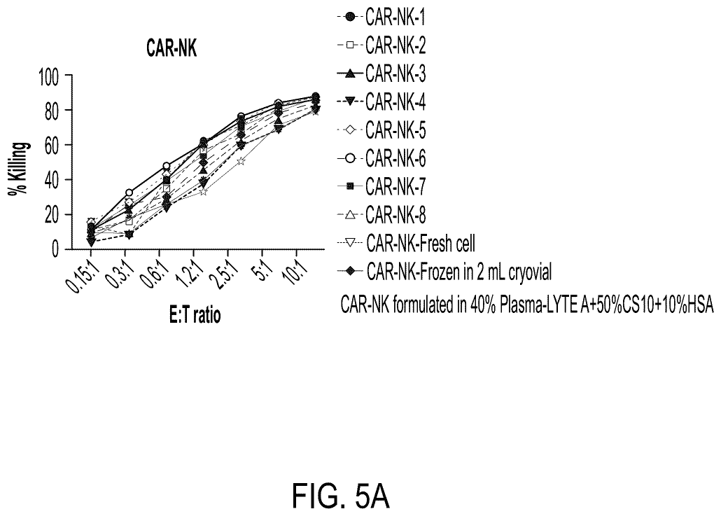

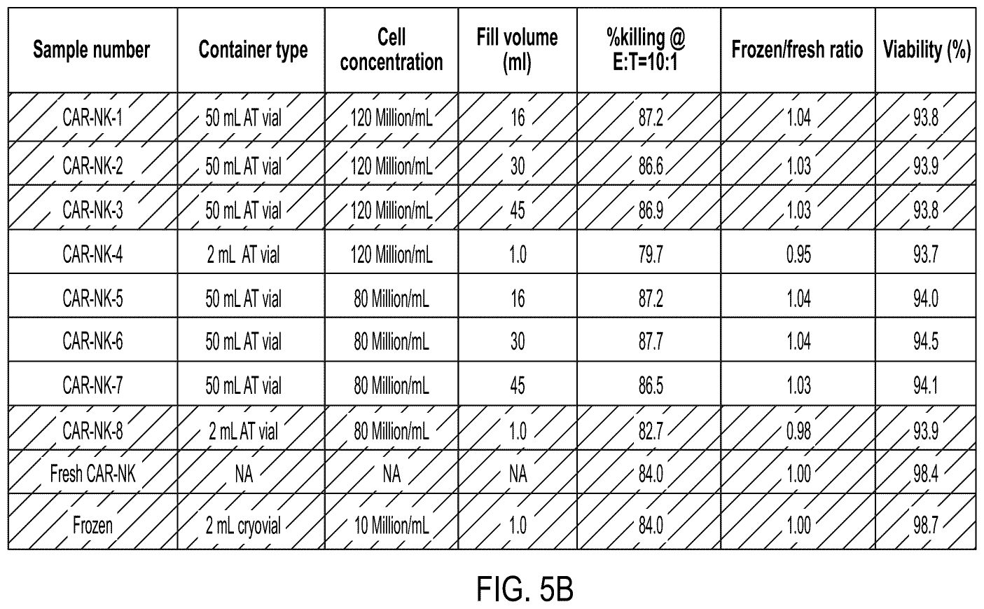

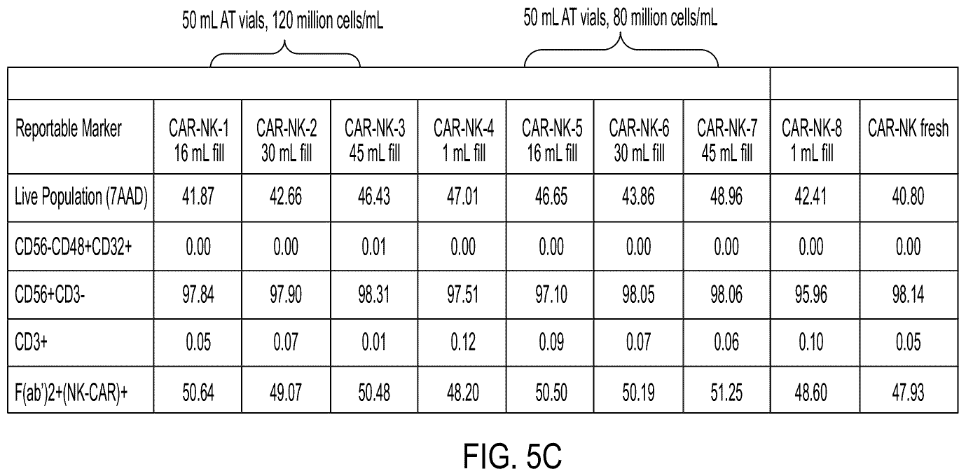

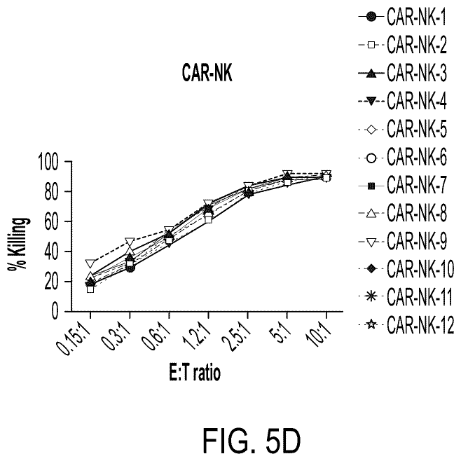

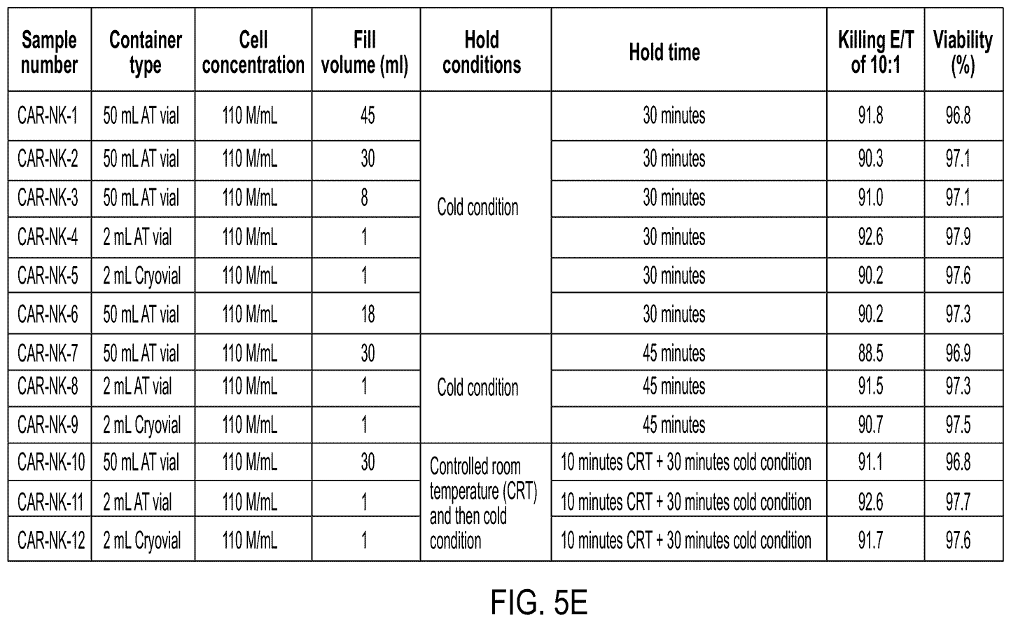

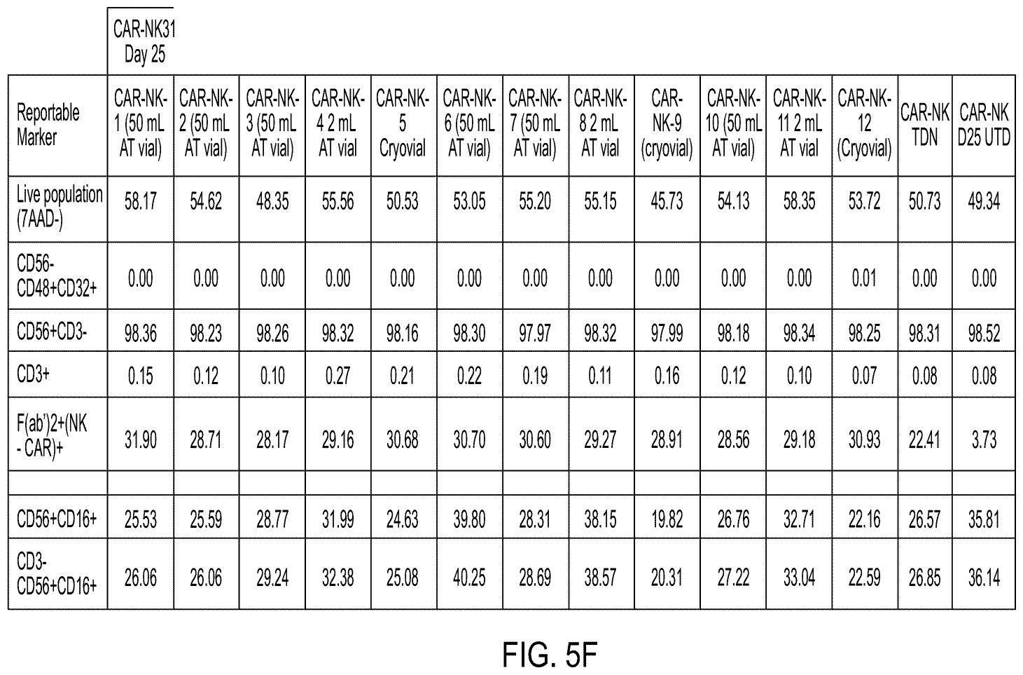

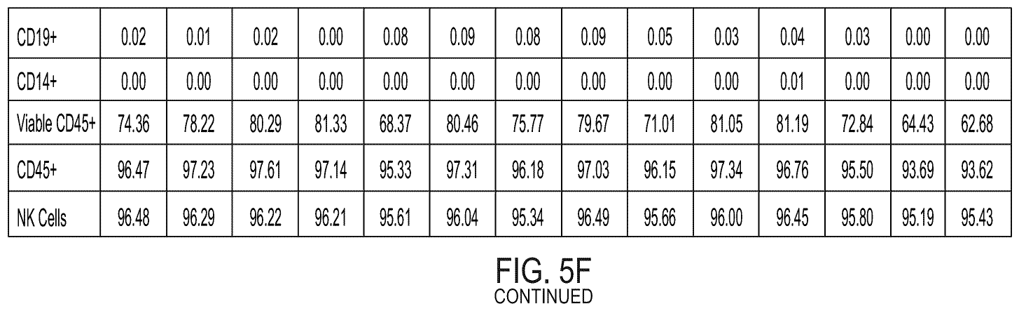

FIG. 5 illustrate the in vitro efficacy and phenotyping of CAR-NK cells of fresh or cryopreserved CAR-NK cells. The in vitro efficacy of CAR-NK cells have been expressed in terms of killing at different E/T ratio, a quantitative expression of killing efficacy (FIGS. 5A, 5B, 5D and 5E). The cryopreserved CAR-NK cells in one donor at different viable cell concentrations of 10M/mL, 80M/mL and 120M/mL showed comparable in vitro killing efficacy of CAR-NK cells post thawing (FIG. 5A and FIG. 5B) and comparable immunophenotyping (FIG. 5C). The cryopreserved CAR-NK cells in one donor at different fill volume of 1 mL, 8 mL, 18 mL, 30 mL, and 45 mL showed comparable in vitro killing efficacy of CAR-NK cells post thawing (FIG. 5D and FIG. 5E) and comparable immunophenotyping (FIG. 5F).

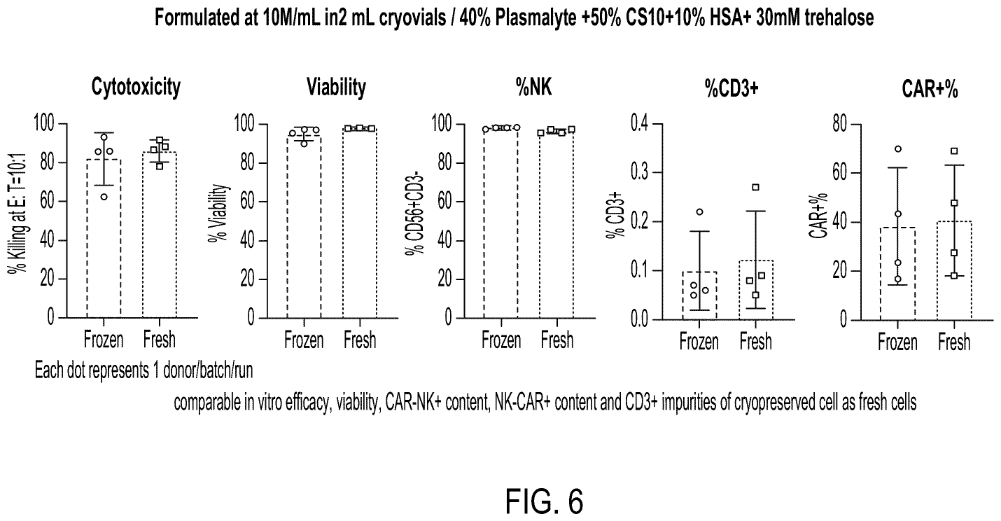

FIG. 6 illustrated the in vitro efficacy (cytotoxicity), viability, phenotyping (% NK, % CD3+ and % CAR+) of CAR-NK cells of fresh are comparable with cryopreserved CAR-NK cells from 4 different donors.

### Example 5: Treating Subject in Need with—CAR-NK—Frozen, Shipped, Thawed, Administered with Vial

This examples describes freezing, thawing and exemplary use of engineered CAR NK cells as described herein. The exemplary use described in this example is the freezing, thawing and the administration of CAR NK cell to a cancer patient, such as a patient who has diffuse large B-cell lymphoma.

CAR-NK cells comprising CD19, IL-15 and iCaspase9 are suspended in a cryopreservation medium comprising human serum albumin (HSA), PLASMA-LYTE A, trehalose and CS10. The CAR-NK cells are frozen at a concentration of 6 M/mL to 25 M/mL in 50 mL cryovials at a fill sample fill volume of about 36 mL. One such cryovial can contain between 2 to 4 doses for a patient in need. The CAR-NK cells are frozen using the following freezing program comprising: (a) placing the sample at a first temperature above the freezing temperature of the sample; (b) reducing the first temperature to a second temperature at a first controlled rate, where the second temperature is at least 2° C. lower than the first temperature; (c) reducing the second temperature to a third temperature at a second controlled rate, where the third temperature is at least 40° C. lower than the second temperature; (d) increasing the third temperature to a fourth temperature at a third controlled rate, where the fourth temperature is at least 20° C. higher than the third temperature; (e) reducing the fourth temperature to a fifth temperature at a fourth controlled rate, where the fifth temperature is at least 10° C. lower than the fourth temperature; and (f) reducing the fifth temperature to the final temperature at a fifth controlled rate, where the final temperature is less than or equal to −80° C. Typically, the entire freezing process takes less than about 1 hour.

Once the cells are frozen, the sample is stored at a temperature of −140° C. or below. Such temperatures can be achieved in various manners, such as placement of the sample in liquid nitrogen vapor phase. The frozen can remain in storage stored at a temperature of −140° C. or below until needed for use. The time the cells can remain in storage for 1 week, 2 weeks, 1 month, 6 months, 1 year, 2 years, 5 years, 10 years or more.

Once the cells are needed for use, such as for example, for use in allogeneic cell therapy, the cells are transported from storage to a hospital or other location in which a patient awaits transplant with the cells. During the shipping process, the cells are maintained at a temperature of −140° C. or below until then reach the hospital or other location. Once at the location, the cells are then thawed. The cells can be thawed as follows: heating a container comprising the cryopreserved engineered immune cells to a temperature of between 37° C. and 70° C.; and agitating the cells at a speed of between about 100 and about 250 RPM for a suitable period of time until the cells are thawed. The heating can be done, for example, at a temperature of between 60° C. and 65° C. while agitating the sample of cells at a speed at between 100 and 125 RPM. The heating can either be performed using a water bath or using a dry heating device. Typically, the entire time to thaw the cells is about 10 minutes.

Thawing of the 50 mL cryovial can be performed at patient's bedside or other nearby location for easy access to the patient who will receive the cells. Once the sample is thawed, the total volume of the sample will be between about 34 to 36 mL. Up to about 34 mL of the thawed sample is administered into a patient using a vial adapter for aseptic administration. One thawed sample may contain multiple doses.

### Equivalents and Scope

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1-45. (canceled)

46. A cell therapy product comprising a CAR-NK cell population comprising NK cells genetically modified to express a CD19 CAR and an IL-15 formulated in a cryopreservation medium comprising PLASMA-LYTE A, trehalose, CS10, and HSA.

47. The cell therapy product of claim 46, wherein the CD19 CAR comprises an anti-CD19 binding domain, a transmembrane domain, and an intracellular signaling domain.

48. The cell therapy product of claim 47, wherein the anti-CD19 binding domain comprises a single chain antibody or fragment thereof.

49. The cell therapy product of claim 46, wherein the CD19 CAR comprises a heavy chain variable region having at least 95% identity to SEQ ID NO: 2 and/or a light chain variable region having at least 95% identity to SEQ ID NO: 1.

50. The cell therapy product of claim 49, wherein the CD19 CAR comprises a heavy chain variable region having the sequence of SEQ ID NO: 2 and/or a light chain variable region having the sequence of SEQ ID NO: 1.

51. The cell therapy product of claim 46, wherein the CD19 CAR comprises a CD28 transmembrane domain having at least 95% identity to SEQ ID NO: 3.

52. The cell therapy product of claim 51, wherein the CD19 CAR comprises a CD28 transmembrane domain of SEQ ID NO: 3.

53. The cell therapy product of claim 46, wherein the CD19 CAR comprises a CD3z signaling domain having at least 95% identity to SEQ ID NO: 4.

54. The cell therapy product of claim 53, wherein the CD19 CAR comprises a CD3z transmembrane domain of SEQ ID NO: 4.

55. The cell therapy product of claim 46, wherein the NK cells are derived from cord-blood, peripheral blood, T cells, or iPS cells.

56. The cell therapy product of claim 46, wherein the cryopreservation medium comprises 25% v/v to 50% v/v PLASMA-LYTE A.

57. The cell therapy product of claim 56, wherein the cryopreservation medium comprises about 40% v/v PLASMA-LYTE A.

58. The cell therapy product of claim 46, wherein the trehalose is at a concentration of between about 10 mM-100 mM.

59. The cell therapy product of claim 46, wherein the HSA is at a concentration of between about 1.25% v/v to 15% v/v of 25% w/v.

60. The cell therapy product of claim 46, wherein the cryopreservation medium comprises 37.7% PLASMA-LYTE A, 30 mM trehalose, 50% CS10, and 2.35% w/v HSA.

61. The cell therapy product of claim 46, further comprising a cryoprotectant, wherein the cryoprotectant is DMSO.

62. A method of treating cancer comprising cryopreserving, thawing and administering to a subject in need thereof, a therapeutically effective amount of a composition comprising a cell therapy product comprising a CAR-NK cell population comprising NK cells genetically modified to express a CD-19 CAR and an IL-15 formulated in a cryopreservation medium comprising PLASMA-LYTE A, trehalose, CS10, and HSA, wherein the CD19 CAR comprises an anti-CD19 binding domain comprising a heavy chain variable region having at least 95% identity to SEQ ID NO: 2 and/or a light chain variable region having at least 95% identity to SEQ ID NO: 1.

63. A cryopreservation medium suitable for immune cells, the medium comprising: PLASMA-LYTE A, human serum albumin (HSA), trehalose, and a cryoprotectant.

64. The cryopreservation medium of claim 63, wherein the cryoprotectant is DMSO.

65. The cryopreservation medium of claim 63, wherein the medium further comprises 0-55 mM HEPES, 0-6% v/v dextran, a sugar alcohol, a metabolite, and an anti-oxidant.

66. The cryopreservation medium of claim 65, wherein the anti-oxidant is glutathione.


SEQUENCE LISTING (4 sequences)

SEQ ID NO: 1 - protein, 108 aa, synthetic construct
  REGION 1..108: note = Anti-CD19 Light chain variable fragment, VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKR

SEQ ID NO: 2 - protein, 130 aa, synthetic construct
  REGION 1..130: note = Anti-CD19 Heavy chain variable fragment, VH
EVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSYVTVSSQDPA

SEQ ID NO: 3 - protein, 68 aa, synthetic construct
  REGION 1..68: note = CD28
FWVLVVVGGVLACYSLLVTVAFIIFWVRSKRSRLLHSDYMNMTPRRPGPTRKHYQPYAPPRDFAAYRS

SEQ ID NO: 4 - protein, 114 aa, synthetic construct
  REGION 1..114: note = CD3-zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGP